(19) United States
(12) Patent Application Publication
(10) Pub. No.: US 2024/0307276 A1
(43) Pub. Date: Sep. 19, 2024

(54) ANTI-POLLUTION AGENT

(71) Applicant: Omya International AG, Oftringen (CH)

(72) Inventors: Tanja BUDDE, Brittnau (CH); Anaïs HECKER, Oftringen (CH)

(73) Assignee: Omya International AG, Oftringen (CH)

(21) Appl. No.: 18/546,770

(22) PCT Filed: Feb. 15, 2022
(86) PCT No.: PCT/EP2022/053640

§ 371 Date: Aug. 16, 2023

(30) Foreign Application Priority Data
Feb. 18, 2021 (EP) 21157958.6

(51) Int. Cl. A61K 8/19; A61K 8/34; A61K 8/891; A61K 8/893; A61K 8/92; A61Q 17/04; A61Q 19/10

(52) U.S. Cl. CPC A61K 8/19; A61K 8/345; A61K 8/891; A61K 8/893; A61K 8/922; A61Q 17/04; A61Q 19/10

(57) ABSTRACT

The present invention is directed to the use of a mineral material as anti-pollution cosmetic agent, wherein the mineral material has a volume median particle size d₅₀(vol) from 0.1 to 90 μm, a volume top cut particle size d₉₈(vol) of below 250 μm, and is selected from surface-reacted calcium carbonate, hydromagnesite, or mixtures thereof. Furthermore, the present invention relates to an anti-pollution cosmetic composition comprising said mineral material as well as a cosmetic method of protecting a keratin material from pollutants comprising the application of said anti-pollution cosmetic composition onto the keratin material.

The present invention relates to anti-pollution agents for cosmetic application, and in particular, to an anti-pollution cosmetic composition comprising a mineral material, the use of said mineral material as anti-pollution cosmetic agent, and a method of protecting a keratin material from pollutions involving the use of said mineral material.

In the last three decades, the pollution of gaseous, aerosol and liquid media such as air, water and soil has become a major environmental concern, especially in urban areas. The sources of environment pollution are diverse, including vehicular traffic and exhausts, coal burning power plants, industrial combustion, cigarette smoke, indoor domestic kitchen cooking fires, and Volatile Organic Compounds. The pollutants in these sources include Particulate Matter (PM), oxides of carbon, sulfur and nitrogen, ozone, free radicals, and other airborne chemicals, like pesticides, chemical sprays, and hydrocarbons (cf. Mistry, Cosmetics 2017, 4, 57).

Pollutants such as nitrogen oxides (NOx) contribute to urban air quality problems, e.g. photochemical smog, and are said to adversely affect the health of human beings as well as of animals and plants. These pollutants are typically emitted in the environment from combustion processes such as power and heating plants, and motor vehicles and/or production processes such as industrial plants. Furthermore, said pollutants are also known as ozone precursors as the major formation of tropospheric ozone results from a reaction of nitrogen oxides (NOx) and volatile organic compounds in the atmosphere in the presence of sunlight and carbon monoxide. Moreover, such reaction may cause photochemical smog, especially in summer time, comprising peroxyacetyl nitrate (PAN) and acid rain. Children, people with lung diseases such as asthma, and people who work or exercise outside are susceptible to adverse effects of photochemical smog such as damage to lung tissue and reduction in lung function.

However, there is growing evidence that environmental pollutants are also harmful to skin. It is widely recognized that pollution can damage skin barrier, result in depletion of vitamin E and squalene level, and breakdown of collagen and elastin exacerbating existing skin problems such as dehydrated skin, hyperpigmentation, photoaging, excessive sebum secretion, inflammation and sensitive skin, eczema, and atopic dermatitis. Moreover, there is evidence that skin quality is impacted by the bad environmental conditions and depending on skin type, people observe an aggravation of their skin problem e.g., dry and dull skin, dark spots and uneven skin tone, wrinkles and fine lines, oily skin and acne, sensitive skin, and imperfection (cf. Mistry, Cosmetics 2017, 4, 57).

Plant extracts, vitamins, or antioxidant complexes are among the most popular anti-pollution ingredients on the market. They are generally used in facial masks or facial skincare. U.S. Pat. No. 5,571,503 A, for example, discloses a cosmetic composition comprising an anti-pollution complex comprising propylene glycol, hydrolyzed wheat protein, mannitol, glycogen, yeast extract, ginseng extract, linden extract, calcium pantothenate, horse chestnut extract, and biotin; a micellar complex comprising: phospholipids, glycosphingolipids, panthenol, cholesterol, Crataegus extract, and sodium hyaluronate; an anti-free radical complex comprising melanin, a short-chain fatty acid ester of tocopherol, a long-chain fatty acid ester of retinol, and a long-chain fatty acid ester of ascorbic acid; and a sunscreen.

WO2018073971 A1 describes a method of protecting keratin material from pollutants, wherein a composition comprising at least one particle having a wet point for oil being at least 100 ml/100 g and a wet point for water being at least 100 ml/100 g is applied to the keratin material. The use of ergothionine and/or its derivates as an anti-pollution cosmetic agent is disclosed in US20040047823 A1.

EP2997833 A1 relates to the use of a surface-reacted calcium carbonate as anti-caking agent. An abrasive cleaning composition comprising at least 6 wt.-%, based on the total weight of the composition, of a surface-reacted calcium carbonate as an abrasive material is described in EP2883573 A1. EP3216510 A1 is concerned with a process for taking up one or more nitrogen oxide(s) from a gaseous and/or aerosol or liquid medium using at least one particulate earth alkali carbonate-comprising material. The use of a surface-reacted calcium carbonate having a volume median particle size from 0.1 to 90 μm as skin appearance modifier in a cosmetic and/or skin care composition is described in EP3517176.

Thus, there is a constant need in the art for cosmetic formulations which can prevent the harmful effects caused by environmental pollutants to the skin.

Accordingly, it is an object of the present invention to provide a cosmetic agent which may protect keratin material such as skin, nail, or hair against environmental pollutants. Furthermore, it would be desirable that the cosmetic agent also provides additional functionality such as a care or cleaning effect. It would also be desirable that the cosmetic agent is derivable from natural resources, is environmentally safe, and easy degradable.

It is also an object of the present invention to provide a cosmetic agent that can serve as source for calcium and/or phosphate ions, which play a role in regulating the skin's functions. Furthermore, it would be desirable that the cosmetic agent is suitable as a carrier material for additional ingredients such as moisturizers or active agents.

Furthermore, it is an object of the present invention to provide a cosmetic composition that can be easily applied to the skin and forms an even and uniform film on the skin. It would also be desirable that the cosmetic composition is less greasy and sticky and provides the skin with a natural and/or mat look. Moreover, it would be desirable that the cosmetic composition exhibits a good spreadability and dries fast.

The foregoing and other objects are solved by the subject-matter as defined in the independent claims.

According to one aspect of the present invention, use of a mineral material as anti-pollution cosmetic agent is provided, wherein the mineral material has
a volume median particle size d₅₀(vol) from 0.1 to 90 μm,
a volume top cut particle size d₉₈(vol) of below 250 μm, and
is selected from surface-reacted calcium carbonate, hydromagnesite, or mixtures thereof,
wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

According to a further aspect of the present invention, an anti-pollution cosmetic composition comprising a mineral material is provided, wherein the mineral material has
a volume median particle size d₅₀(vol) from 0.1 to 90 μm,
a volume top cut particle size d₉₈(vol) of below 250 μm, and
is selected from surface-reacted calcium carbonate, hydromagnesite, or mixtures thereof,
wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

According to still a further aspect of the present invention, a cosmetic method of protecting a keratin material from pollutants is provided, the method comprising:
(i) providing a anti-pollution cosmetic composition according to the present invention, and
(ii) applying said anti-pollution cosmetic composition onto the keratin material.

Advantageous embodiments of the present invention are defined in the corresponding subclaims.

According to one embodiment the mineral material has a volume median particle size d₅₀(vol) from 0.1 to 75 μm, preferably from 0.5 to 50 μm, more preferably from 1 to 40 μm, even more preferably from 1.2 to 30 μm, and most preferably from 1.5 to 15 μm, and/or a volume top cut particle size d₉₈(vol) from 0.2 to 150 μm, preferably from 1 to 100 μm, more preferably from 2 to 80 μm, even more preferably from 2.4 to 60 μm, and most preferably from 3 to 30 μm. According to another embodiment the mineral material has an intra-particle intruded specific pore volume in the range from 0.05 to 2.3 cm3/g, preferably from 0.1 to 2.0 cm3/g, more preferably from 0.2 to 2.5 cm3/g and most preferably from 0.3 to 2.2 cm3/g, calculated from a mercury porosimetry measurement, for a diameter range of 0.004 to 0.8 μm. According to still another embodiment the mineral material has a specific surface area of from 15 m2/g to 200 m2/g, preferably from 20 m2/g to 180 m2/g, more preferably from 25 m2/g to 160 m2/g, even more preferably from 27 m2/g to 150 m2/g, and most preferably from 30 m2/g to 140 m2/g, measured using nitrogen and the BET method.

According to one embodiment the natural ground calcium carbonate is selected from the group consisting of marble, chalk, limestone, and mixtures thereof, or the precipitated calcium carbonate is selected from the group consisting of precipitated calcium carbonates having an aragonitic, vateritic or calcitic crystal form, and mixtures thereof. According to another embodiment the at least one H₃O⁺ ion donor is selected from the group consisting of hydrochloric acid, sulphuric acid, sulphurous acid, phosphoric acid, citric acid, oxalic acid, an acidic salt, acetic acid, formic acid, and mixtures thereof, preferably the at least one H₃O⁺ ion donor is selected from the group consisting of hydrochloric acid, sulphuric acid, sulphurous acid, phosphoric acid, oxalic acid, H₂PO₄⁻, being at least partially neutralised by a cation selected from Li⁺, Na⁺ and/or K⁺, HPO₄²⁻, being at least partially neutralised by a cation selected from Li⁺, Nat, K⁺, Mg²⁺, and/or Ca²⁺, and mixtures thereof, more preferably the at least one H₃O⁺ ion donor is selected from the group consisting of hydrochloric acid, sulphuric acid, sulphurous acid, phosphoric acid, oxalic acid, or mixtures thereof, and most preferably, the at least one H₃O⁺ ion donor is phosphoric acid. According to still another embodiment the mineral material is associated with at least one active agent selected from pharmaceutically active agents, biologically active agents, disinfecting agents, preservatives, flavouring agents, surfactants, oils, fragrances, essential oils, and mixtures thereof.

According to one embodiment the mineral material is present in the anti-pollution cosmetic composition in an amount from 0.1 to 50 wt.-%, based on the total weight of the anti-pollution cosmetic composition, preferably from 0.5 to 20 wt.-%, more preferably from 1 to 10 wt.-%, and most preferably from 3 to 6 wt.-%. According to another embodiment the anti-pollution cosmetic composition has a pH value of ≤8.5, preferably ≤8.0, more preferably ≤7.5, even more preferably ≤7.0, and most preferably from 4.0 to 7.0.

According to one embodiment the anti-pollution cosmetic composition further comprises water and/or at least one oil, preferably the at least one oil is selected from the group consisting of vegetable oils and esters thereof, alkanecoconutester, plant extracts, animal fats, siloxanes, silicones, fatty acids and esters thereof, petrolatum, glycerides and pegylated derivatives thereof, and mixtures thereof. According to another embodiment the anti-pollution cosmetic composition further comprises at least one additive selected from the group consisting of bleaching agents, thickeners, stabilizers, chelating agents, preserving agents, wetting agents, emulsifiers, emollients, fragrances, colorants, skin tanning compounds, antioxidants, minerals, pigments, UV-A and/or UV-B filter, and mixtures thereof. According to still another embodiment the anti-pollution cosmetic composition is a sun protection product, an eye make-up product, a facial make-up product, a lip care product, a hair care product, a hair styling product, a hair cleaning product, a nail care product, a hand care product, a hand cleaning product, a skin care product, a skin cleaning product, a scalp care product, a scalp cleaning product, a facial cleaning product, a make-up remover, a facial mist, a cleaning wipe, an exfoliating product, or a combination product thereof.

According to one embodiment the pollutants are atmospheric pollutants, preferably selected from the group consisting of carbon black, carbon oxides, nitrogen oxides, sulfur oxides, hydrocarbons, organic volatiles, heavy metals, atmospheric particulate matter, fine particulate matter (PM_{2.5}), and mixtures thereof, and/or wherein the keratin material is skin, nails, and/or hair.

It should be understood that for the purpose of the present invention, the following terms have the following meaning:

The term “acid” as used herein refers to an acid in the meaning of the definition by Brønsted and Lowry (e.g., H₂SO₄, HSO₄⁻), wherein the term “free acid” refers only to those acids being in the fully protonated form (e.g., H₂SO₄).

The term “aqueous” suspension refers to a system, wherein the liquid phase comprises, preferably consists of, water. However, said term does not exclude that the liquid phase of the aqueous suspension comprises minor amounts of at least one water-miscible organic solvent selected from the group comprising methanol, ethanol, acetone, acetonitrile, tetrahydrofuran and mixtures thereof. If the aqueous suspension comprises at least one water-miscible organic solvent, the liquid phase of the aqueous suspension comprises the at least one water-miscible organic solvent in an amount of from 0.1 to 40.0 wt.-% preferably from 0.1 to 30.0 wt.-%, more preferably from 0.1 to 20.0 wt.-% and most preferably from 0.1 to 10.0 wt.-%, based on the total weight of the liquid phase of the aqueous suspension. For example, the liquid phase of the aqueous suspension consists of water.

For the purpose of the present invention a “cosmetic agent” or a “cosmetic composition”, respectively, refers to any substance or mixture, respectively, intended to be placed in contact with the various external parts of the human body (epidermis, hair system, nails, lips and external genital organs) with a view exclusively or mainly to cleaning them, perfuming them, changing their appearance and/or correcting body odours and/or protecting them or keeping them in good condition (cf. EU Cosmetics Regulation, Article 2 (Regulation (EC) No. 1223/2009)). In the meaning the present invention, the term “cosmetic” does not encompasses a therapeutic use, but merely refers to a non-therapeutic use.

Unless specified otherwise, the term “drying” refers to a process according to which at least a portion of water is removed from a material to be dried such that a constant weight of the obtained “dried” material at 200° C. is reached. Moreover, a “dried” or “dry” material may be defined by its total moisture content which, unless specified otherwise, is less than or equal to 1.0 wt.-%, preferably less than or equal to 0.5 wt.-%, more preferably less than or equal to 0.2 wt.-%, and most preferably between 0.03 and 0.07 wt.-%, based on the total weight of the dried material.

For the purpose of the present invention, the term “hydromagnesite” refers to a mineral material having the chemical composition of Mg₅(CO₃)₄(OH)₂·4H₂O.

“Natural ground calcium carbonate” (GCC) in the meaning of the present invention is a calcium carbonate obtained from natural sources, such as limestone, marble, or chalk, and processed through a wet and/or dry treatment such as grinding, screening and/or fractionating, for example, by a cyclone or classifier.

“Precipitated calcium carbonate” (PCC) in the meaning of the present invention is a synthesised material, obtained by precipitation following reaction of carbon dioxide and lime in an aqueous, semi-dry or humid environment or by precipitation of a calcium and carbonate ion source in water. PCC may be in the vateritic, calcitic or aragonitic crystal form. PCCs are described, for example, in EP2447213 A1, EP2524898 A1, EP2371766 A1, EP1712597 A1, EP1712523 A1, or WO2013142473 A1.

The “particle size” of particulate materials other than surface-reacted calcium carbonate and hydromagnesite herein is described by its weight-based distribution of particle sizes dx. Therein, the value dx represents the diameter relative to which x % by weight of the particles have diameters less than dx. This means that, for example, the d₂₀ value is the particle size at which 20 wt.-% of all particles are smaller than that particle size. The d₅₀ value is thus the weight median particle size, i.e. 50 wt.-% of all particles are smaller than this particle size. For the purpose of the present invention, the particle size is specified as weight median particle size d₅₀(wt) unless indicated otherwise. Particle sizes were determined by using a Sedigraph™ 5100 instrument or Sedigraph™ 5120 instrument of Micromeritics Instrument Corporation. The method and the instrument are known to the skilled person and are commonly used to determine the particle size of fillers and pigments. The measurements were carried out in an aqueous solution of 0.1 wt.-% Na₄P₂O₇.

The “particle size” of surface-reacted calcium carbonate and hydromagnesite herein is described as volume-based particle size distribution. The volume median particle size d₅₀(vol) or the volume top cut particle size d₉₈(vol), measured using a Malvern Mastersizer 2000 or 3000 Laser Diffraction System, indicates a diameter value such that 50% or 98% by volume, respectively, of the particles have a diameter of less than this value. The raw data obtained by the measurement are analysed using the Mie theory, with a particle refractive index of 1.57 and an absorption index of 0.005.

“Pollutant” in the meaning of the present invention refers to a substance that is present in concentrations that may harm organisms (humans, plants and animals) or exceed an environmental quality standard. In particular, the term “pollutant” may refer to an atmospheric pollutant such as particulate matter (PM), especially fine particulate matter (PM_{2.5}), carbon black, carbon oxides, nitrogen oxides, sulfur oxides, hydrocarbons, organic volatiles, or heavy metals. Accordingly, the expression “anti-pollution” cosmetic agent or cosmetic composition refers to an cosmetic agent or cosmetic composition that can protect a keratin material, e.g. skin, nail and/or hair, from a pollutant.

In the context of the present invention, the term “pore” is to be understood as describing the space that is found between and/or within particles, i.e. that is formed by the particles as they pack together under nearest neighbor contact (inter-particle pores), such as in a powder or a compact and/or the void space within porous particles (intra-particle pores), and that allows the passage of liquids under pressure when saturated by the liquid and/or supports absorption of surface wetting liquids.

The specific pore volume is measured using a mercury intrusion porosimetry measurement using a Micromeritics Autopore V 9620 mercury porosimeter having a maximum applied pressure of mercury 414 MPa (60 000 psi), equivalent to a Laplace throat diameter of 0.004 μm (˜nm). The equilibration time used at each pressure step is 20 seconds. The sample material is sealed in a 3 cm3 chamber powder penetrometer for analysis. The data are corrected for mercury compression, penetrometer expansion and sample material compression using the software Pore-Comp (Gane, P. A. C., Kettle, J. P., Matthews, G. P. and Ridgway, C. J., “Void Space Structure of Compressible Polymer Spheres and Consolidated Calcium Carbonate Paper-Coating Formulations”, Industrial and Engineering Chemistry Research, 35(5), 1996, p. 1753-1764.).

The total pore volume seen in the cumulative intrusion data can be separated into two regions with the intrusion data from 214 μm down to about 1-4 μm showing the coarse packing of the sample between any agglomerate structures contributing strongly. Below these diameters lies the fine inter-particle packing of the particles themselves. If they also have intra-particle pores, then this region appears bimodal, and by taking the specific pore volume intruded by mercury into pores finer than the modal turning point, i.e. finer than the bimodal point of inflection, we thus define the specific intra-particle pore volume. The sum of these three regions gives the total overall pore volume of the powder, but depends strongly on the original sample compaction/settling of the powder at the coarse pore end of the distribution.

By taking the first derivative of the cumulative intrusion curve the pore size distributions based on equivalent Laplace diameter, inevitably including pore-shielding, are revealed. The differential curves clearly show the coarse agglomerate pore structure region, the inter-particle pore region and the intra-particle pore region, if present. Knowing the intra-particle pore diameter range it is possible to subtract the remainder inter-particle and inter-agglomerate pore volume from the total pore volume to deliver the desired pore volume of the internal pores alone in terms of the pore volume per unit mass (specific pore volume). The same principle of subtraction, of course, applies for isolating any of the other pore size regions of interest.

A “salt” in the meaning of the present invention is a chemical compound consisting of an assembly of cations and anions (cf. IUPAC, Compendium of Chemical Terminology, 2^{nd} Ed. (the “gold book”), 1997, “salt”).

For the purpose of the present invention, the “solids content” of a liquid composition is a measure of the amount of material remaining after all the solvent or water has been evaporated. If necessary, the “solids content” of a suspension given in wt.-% in the meaning of the present invention can be determined using a Moisture Analyzer HR73 from Mettler-Toledo (T=120° C., automatic switch off 3, standard drying) with a sample size of 5 to 20 g.

The “specific surface area” (expressed in m2/g) of a material as used throughout the present document can be determined by the Brunauer Emmett Teller (BET) method with nitrogen as adsorbing gas and by use of a ASAP 2460 instrument from Micromeritics. The method is well known to the skilled person and defined in ISO 9277:2010. Samples are conditioned at 100° C. under vacuum for a period of 30 min prior to measurement. The total surface area (in m2) of said material can be obtained by multiplication of the specific surface area (in m2/g) and the mass (in g) of the material.

The term “surface-reacted” in the meaning of the present application shall be used to indicate that a material has been subjected to a process comprising partial dissolution of said material in aqueous environment followed by a crystallization process on and around the surface of said material, which may occur in the absence or presence of further crystallization additives.

A “suspension” or “slurry” in the meaning of the present invention comprises undissolved solids and water, and optionally further additives, and usually contains large amounts of solids and, thus, is more viscous and can be of higher density than the liquid from which it is formed.

For the purpose of the present invention, the term “viscosity” or “Brookfield viscosity” refers to Brookfield viscosity. The Brookfield viscosity is for this purpose measured by a Brookfield DV-II+ Pro viscometer at 25° C.±1° C. at 100 rpm using an appropriate spindle of the Brookfield RV-spindle set and is specified in mPa·s. Based on his technical knowledge, the skilled person will select a spindle from the Brookfield RV-spindle set which is suitable for the viscosity range to be measured. For example, for a viscosity range between 200 and 800 mPa·s the spindle number 3 may be used, for a viscosity range between 400 and 1 600 mPa's the spindle number 4 may be used, for a viscosity range between 800 and 3 200 mPa·s the spindle number 5 may be used, for a viscosity range between 1 000 and 2 000 000 mPa·s the spindle number 6 may be used, and for a viscosity range between 4 000 and 8 000 000 mPa·s the spindle number 7 may be used.

For the purpose of the present application, “water-insoluble” materials are defined as materials which, when 100 g of said material is mixed with 100 g deionised water and filtered on a filter having a 0.2 μm pore size at 20° C. to recover the liquid filtrate, provide less than or equal to 1 g of recovered solid material following evaporation at 95 to 100° C. of 100 g of said liquid filtrate at ambient pressure. “Water-soluble” materials are defined as materials which, when 100 g of said material is mixed with 100 g deionised water and filtered on a filter having a 0.2 μm pore size at 20° C. to recover the liquid filtrate, provide more than 1 g of recovered solid material following evaporation at 95 to 100° C. of 100 g of said liquid filtrate at ambient pressure.

Where an indefinite or definite article is used when referring to a singular noun, e.g., “a”, “an” or “the”, this includes a plural of that noun unless anything else is specifically stated.

Where the term “comprising” is used in the present description and claims, it does not exclude other elements. For the purposes of the present invention, the term “consisting of” is considered to be a preferred embodiment of the term “comprising”. If hereinafter a group is defined to comprise at least a certain number of embodiments, this is also to be understood to disclose a group, which preferably consists only of these embodiments.

Terms like “obtainable” or “definable” and “obtained” or “defined” are used interchangeably. This, for example, means that, unless the context clearly dictates otherwise, the term “obtained” does not mean to indicate that, for example, an embodiment must be obtained by, for example, the sequence of steps following the term “obtained” though such a limited understanding is always included by the terms “obtained” or “defined” as a preferred embodiment.

Whenever the terms “including” or “having” are used, these terms are meant to be equivalent to “comprising” as defined hereinabove.

According to the present invention, use of a mineral material as anti-pollution cosmetic agent is provided. The mineral material has a volume median particle size d₅₀(vol) from 0.1 to 90 μm, a volume top cut particle size d₉₈(vol) of below 250 μm, and is selected from surface-reacted calcium carbonate, hydromagnesite, or mixtures thereof. The surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

In the following preferred embodiment of the inventive use will be set out in more detail. It is to be understood that these embodiments and details also apply to the inventive composition and the inventive method.

### Mineral Material

According to the present invention, a mineral material is used as anti-pollution cosmetic agent, wherein the material is selected from surface-reacted calcium carbonate, hydromagnesite, or mixtures thereof. The mineral material has a volume median particle size d₅₀(vol) from 0.1 to 90 μm, and a volume top cut particle size d₉₈(vol) of below 250 μm.

According to one embodiment, the mineral material has a volume median particle size d₅₀(vol) from 0.1 to 75 μm, preferably from 0.5 to 50 μm, more preferably from 1 to 40 μm, even more preferably from 1.2 to 30 μm, and most preferably from 1.5 to 15 μm. In addition or alternatively, the mineral material may have a volume top cut particle size d₉₈(vol) from 0.2 to 150 μm, preferably from 1 to 100 μm, more preferably from 2 to 80 μm, even more preferably from 2.4 to 60 μm, and most preferably from 3 to 30 μm.

The value dx represents the diameter relative to which x % of the particles have diameters less than dx. This means that the dos value is the particle size at which 98% of all particles are smaller. The dos value is also designated as “top cut”. The dx values may be given in volume or weight percent. The d₅₀ (wt) value is thus the weight median particle size, i.e. 50 wt.-% of all grains are smaller than this particle size, and the d₅₀ (vol) value is the volume median particle size, i.e. 50 vol.-% of all grains are smaller than this particle size.

Volume median particle size d₅₀(vol) and volume top cut particle size d₉₈(vol) are evaluated using a Malvern Mastersizer 2000 Laser Diffraction System. The d₅₀ or dos value, measured using a Malvern Mastersizer 2000 Laser Diffraction System, indicates a diameter value such that 50% or 98% by volume, respectively, of the particles have a diameter of less than this value. The raw data obtained by the measurement are analysed using the Mie theory, with a particle refractive index of 1.57 and an absorption index of 0.005.

The weight median particle size d₅₀(wt) and weight top cut particle size d₉₈(wt) are determined by the sedimentation method, which is an analysis of sedimentation behaviour in a gravimetric field. The measurement is made with a Sedigraph™ 5100 or 5120, Micromeritics Instrument Corporation. The method and the instrument are known to the skilled person and are commonly used to determine grain size of fillers and pigments. The measurement is carried out in an aqueous solution of 0.1 wt.-% Na₄P₂O₇. The samples were dispersed using a high speed stirrer and sonicated.

The processes and instruments are known to the skilled person and are commonly used to determine particle size of fillers and pigments.

According to one embodiment, the mineral material has a specific surface area of from 15 m²/g to 200 m²/g, preferably from 20 m²/g to 180 m²/g, more preferably from 25 m²/g to 160 m²/g, even more preferably from 27 m²/g to 150 m²/g, and most preferably from 30 m²/g to 140 m²/g, measured using nitrogen and the BET method. The BET specific surface area in the meaning of the present invention is defined as the surface area of the particles divided by the mass of the particles. As used therein the specific surface area is measured by adsorption using the BET isotherm (ISO 9277:2010) and is specified in m²/g.

The specific pore volume is measured using a mercury intrusion porosimetry measurement using a Micromeritics Autopore V 9620 mercury porosimeter having a maximum applied pressure of mercury 414 MPa (60 000 psi), equivalent to a Laplace throat diameter of 0.004 μm (˜ nm). The equilibration time used at each pressure step is 20 seconds. The sample material is sealed in a 5 cm3 chamber powder penetrometer for analysis. The data are corrected for mercury compression, penetrometer expansion and sample material compression using the software Pore-Comp (Gane, P. A. C., Kettle, J. P., Matthews, G. P. and Ridgway, C. J., “Void Space Structure of Compressible Polymer Spheres and Consolidated Calcium Carbonate Paper-Coating Formulations”, Industrial and Engineering Chemistry Research, 35(5), 1996, p 1753-1764.).

The total pore volume seen in the cumulative intrusion data can be separated into two regions with the intrusion data from 214 μm down to about 1-4 μm showing the coarse packing of the sample between any agglomerate structures contributing strongly. Below these diameters lies the fine interparticle packing of the particles themselves. If they also have intraparticle pores, then this region appears bi modal, and by taking the specific pore volume intruded by mercury into pores finer than the modal turning point, i.e. finer than the bi-modal point of inflection, the specific intraparticle pore volume is defined. The sum of these three regions gives the total overall pore volume of the powder, but depends strongly on the original sample compaction/settling of the powder at the coarse pore end of the distribution.

By taking the first derivative of the cumulative intrusion curve the pore size distributions based on equivalent Laplace diameter, inevitably including pore-shielding, are revealed. The differential curves clearly show the coarse agglomerate pore structure region, the interparticle pore region and the intraparticle pore region, if present. Knowing the intraparticle pore diameter range it is possible to subtract the remainder interparticle and interagglomerate pore volume from the total pore volume to deliver the desired pore volume of the internal pores alone in terms of the pore volume per unit mass (specific pore volume). The same principle of subtraction, of course, applies for isolating any of the other pore size regions of interest.

According to one embodiment, the mineral material has an intra-particle intruded specific pore volume in the range from 0.05 to 2.3 cm3/g, preferably from 0.1 to 2.0 cm3/g, more preferably from 0.2 to 2.5 cm3/g and most preferably from 0.3 to 2.2 cm3/g, calculated from a mercury porosimetry measurement, for a diameter range of 0.004 to 0.8 μm.

The intra-particle pore size of the mineral material preferably may be in a range of from 0.004 to 1.5 μm, more preferably in a range of from 0.005 to 1.0 μm, especially preferably from 0.006 to 0.8 μm and most preferably of 0.007 to 0.6 μm, e.g. 0.1 to 0.4, μm determined by mercury porosimetry measurement.

Due to the intra and interpore structure of the mineral material, it can be a superior agent to deliver previously adsorbed and/or absorbed materials over time relative to common materials having similar specific surface areas. Thus, generally, any agent fitting into the intra- and/or inter particle pores of the mineral material is suitable to be transported by the mineral material according to the invention. For example, active agents such as those selected from the group comprising pharmaceutically active agents, biologically active agents, vitamins, disinfecting agents, preservatives, flavouring agents, surfactants, oils, fragrances, essential oils, scented oils, and mixtures thereof can be used. According to one embodiment, at least one active agent is associated with the mineral material, and preferably the mineral material is associated with at least one active agent selected from pharmaceutically active agents, biologically active agents, vitamins, disinfecting agents, preservatives, flavouring agents, surfactants, oils, fragrances, essential oils, scented oils, and mixtures thereof.

The at least one active agent may be adsorbed onto and/or absorbed into the surface of the mineral material in specific amounts. According to one embodiment of the present invention, the amount of the at least one agent being adsorbed onto and/or absorbed into the surface of the mineral material ranges from 0.1 to 99 wt.-%, based on the total weight of the mineral material, preferably ranges from 30 to 95 wt.-%, more preferably from 50 to 90 wt.-%, and most preferably from 70 to 85 wt.-%.

According to one embodiment, the mineral material has a volume median particle size d₅₀(vol) from 1 to 15 μm, a volume top cut particle size d₉₈(vol) from 3 to 30 μm, a specific surface area from 30 m²/g to 100 m²/g, measured using nitrogen and BET method, and an intra-particle intruded specific pore volume in the range from 0.3 to 2.2 cm3/g, calculated from a mercury porosimetry measurement, for a diameter range of 0.004 to 0.8 μm.

According to one embodiment, the mineral material is hydromagnesite and has a volume median particle size d₅₀(vol) from 5 to 8 μm, a volume top cut particle size d₉₈(vol) from 20 to 30 μm, a specific surface area from 30 m²/g to 60 m²/g, measured using nitrogen and BET method, and an intra-particle intruded specific pore volume in the range from 1.8 to 2.2 cm3/g, calculated from a mercury porosimetry measurement, for a diameter range of 0.004 to 0.8 μm.

According to another embodiment, the mineral material is surface-reacted calcium carbonate, wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source, and has a volume median particle size d₅₀(vol) from 3 to 8 μm, a volume top cut particle size d₉₈(vol) from 6 to 15 μm, a specific surface area from 50 m²/g to 100 m²/g, measured using nitrogen and BET method, and an intra-particle intruded specific pore volume in the range from 0.8 to 1.7 cm3/g, calculated from a mercury porosimetry measurement, for a diameter range of 0.004 to 0.8 μm. Preferably the surface-reacted calcium carbonate is a reaction product of natural ground calcium carbonate with carbon dioxide and phosphoric acid.

### Surface-Reacted Calcium Carbonate

According to one embodiment, the mineral material is surface-reacted calcium carbonate and/or a mixture of surface-reacted calcium carbonate and hydromagnesite, wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source. According to another embodiment, the mineral material is surface-reacted calcium carbonate as defined herein. According to still another embodiment, the mineral material is a mixture of surface-reacted calcium carbonate as defined herein and hydromagnesite.

An H₃O⁺ ion donor in the context of the present invention is a Brønsted acid and/or an acid salt.

In a preferred embodiment of the invention the surface-reacted calcium carbonate is obtained by a process comprising the steps of: (a) providing a suspension of natural or precipitated calcium carbonate, (b) adding at least one acid having a pKₐ value of 0 or less at 20° C. or having a pKₐ value from 0 to 2.5 at 20° C. to the suspension of step (a), and (c) treating the suspension of step (a) with carbon dioxide before, during or after step (b). According to another embodiment the surface-reacted calcium carbonate is obtained by a process comprising the steps of: (A) providing a natural or precipitated calcium carbonate, (B) providing at least one water-soluble acid, (C) providing gaseous CO₂, (D) contacting said natural or precipitated calcium carbonate of step (A) with the at least one acid of step (B) and with the CO₂ of step (C), characterised in that: (i) the at least one acid of step B) has a pKₐ of greater than 2.5 and less than or equal to 7 at 20° C., associated with the ionisation of its first available hydrogen, and a corresponding anion is formed on loss of this first available hydrogen capable of forming a water-soluble calcium salt, and (ii) following contacting the at least one acid with natural or precipitated calcium carbonate, at least one water-soluble salt, which in the case of a hydrogen-containing salt has a pKₐ of greater than 7 at 20° C., associated with the ionisation of the first available hydrogen, and the salt anion of which is capable of forming water-insoluble calcium salts, is additionally provided.

“Natural ground calcium carbonate” (GCC) preferably is selected from calcium carbonate containing minerals selected from the group comprising marble, chalk, limestone and mixtures thereof. Natural calcium carbonate may comprise further naturally occurring components such as magnesium carbonate, alumino silicate etc.

In general, the grinding of natural ground calcium carbonate may be a dry or wet grinding step and may be carried out with any conventional grinding device, for example, under conditions such that comminution predominantly results from impacts with a secondary body, i.e. in one or more of: a ball mill, a rod mill, a vibrating mill, a roll crusher, a centrifugal impact mill, a vertical bead mill, an attrition mill, a pin mill, a hammer mill, a pulveriser, a shredder, a de-clumper, a knife cutter, or other such equipment known to the skilled man. In case the calcium carbonate containing mineral material comprises a wet ground calcium carbonate containing mineral material, the grinding step may be performed under conditions such that autogenous grinding takes place and/or by horizontal ball milling, and/or other such processes known to the skilled man. The wet processed ground calcium carbonate containing mineral material thus obtained may be washed and dewatered by well-known processes, e.g. by flocculation, filtration or forced evaporation prior to drying. The subsequent step of drying (if necessary) may be carried out in a single step such as spray drying, or in at least two steps. It is also common that such a mineral material undergoes a beneficiation step (such as a flotation, bleaching or magnetic separation step) to remove impurities.

“Precipitated calcium carbonate” (PCC) in the meaning of the present invention is a synthesized material, generally obtained by precipitation following reaction of carbon dioxide and calcium hydroxide in an aqueous environment or by precipitation of calcium and carbonate ions, for example CaCl₂) and Na₂CO₃, out of solution. Further possible ways of producing PCC are the lime soda process, or the Solvay process in which PCC is a by-product of ammonia production. Precipitated calcium carbonate exists in three primary crystalline forms: calcite, aragonite and vaterite, and there are many different polymorphs (crystal habits) for each of these crystalline forms. Calcite has a trigonal structure with typical crystal habits such as scalenohedral (S-PCC), rhombohedral (R-PCC), hexagonal prismatic, pinacoidal, colloidal (C-PCC), cubic, and prismatic (P-PCC). Aragonite is an orthorhombic structure with typical crystal habits of twinned hexagonal prismatic crystals, as well as a diverse assortment of thin elongated prismatic, curved bladed, steep pyramidal, chisel shaped crystals, branching tree, and coral or worm-like form. Vaterite belongs to the hexagonal crystal system. The obtained PCC slurry can be mechanically dewatered and dried.

According to one embodiment of the present invention, the precipitated calcium carbonate is precipitated calcium carbonate, preferably comprising aragonitic, vateritic or calcitic mineralogical crystal forms or mixtures thereof.

Precipitated calcium carbonate may be ground prior to the treatment with carbon dioxide and at least one H₃O⁺ ion donor by the same means as used for grinding natural calcium carbonate as described above.

The natural and/or precipitated calcium carbonate may be used dry or suspended in water. Preferably, a corresponding slurry has a content of natural or precipitated calcium carbonate within the range of 1 wt.-% to 90 wt.-%, more preferably 3 wt.-% to 60 wt.-%, even more preferably 5 wt.-% to 40 wt.-%, and most preferably 10 wt.-% to 25 wt.-% based on the weight of the slurry.

The one or more H₃O⁺ ion donor used for the preparation of surface reacted calcium carbonate may be any strong acid, medium-strong acid, or weak acid, or mixtures thereof, generating H₃O⁺ ions under the preparation conditions. According to the present invention, the at least one H₃O⁺ ion donor can also be an acidic salt, generating H₃O⁺ ions under the preparation conditions.

According to one embodiment, the at least one H₃O⁺ ion donor is a strong acid having a pKₐ of 0 or less at 20° C.

According to another embodiment, the at least one H₃O⁺ ion donor is a medium-strong acid having a pKₐ value from 0 to 2.5 at 20° C. If the pKₐ at 20° C. is 0 or less, the acid is preferably selected from sulphuric acid, hydrochloric acid, or mixtures thereof. If the pKₐ at 20° C. is from 0 to 2.5, the H₃O⁺ ion donor is preferably selected from H₂SO₃, H₃PO₄, oxalic acid, or mixtures thereof. The at least one H₃O⁺ ion donor can also be an acidic salt, for example, HSO₄⁻ or H₂PO₄⁻, being at least partially neutralized by a corresponding cation such as Li⁺, Na⁺ or K⁺, or HPO₄²⁻, being at least partially neutralised by a corresponding cation such as Li⁺, Na⁺, K⁺, Mg²⁺ or Ca²⁺. The at least one H₃O⁺ ion donor can also be a mixture of one or more acids and one or more acidic salts.

According to still another embodiment, the at least one H₃O⁺ ion donor is a weak acid having a pKₐ value of greater than 2.5 and less than or equal to 7, when measured at 20° C., associated with the ionisation of the first available hydrogen, and having a corresponding anion, which is capable of forming water-soluble calcium salts. Subsequently, at least one water-soluble salt, which in the case of a hydrogen-containing salt has a pKₐ of greater than 7, when measured at 20° C., associated with the ionisation of the first available hydrogen, and the salt anion of which is capable of forming water-insoluble calcium salts, is additionally provided. According to the preferred embodiment, the weak acid has a pKₐ value from greater than 2.5 to 5 at 20° C., and more preferably the weak acid is selected from the group consisting of acetic acid, formic acid, propanoic acid, and mixtures thereof. Exemplary cations of said water-soluble salt are selected from the group consisting of potassium, sodium, lithium and mixtures thereof. In a more preferred embodiment, said cation is sodium or potassium. Exemplary anions of said water-soluble salt are selected from the group consisting of phosphate, dihydrogen phosphate, monohydrogen phosphate, oxalate, silicate, mixtures thereof and hydrates thereof. In a more preferred embodiment, said anion is selected from the group consisting of phosphate, dihydrogen phosphate, monohydrogen phosphate, mixtures thereof and hydrates thereof. In a most preferred embodiment, said anion is selected from the group consisting of dihydrogen phosphate, monohydrogen phosphate, mixtures thereof and hydrates thereof. Water-soluble salt addition may be performed dropwise or in one step. In the case of drop wise addition, this addition preferably takes place within a time period of 10 minutes. It is more preferred to add said salt in one step.

According to one embodiment of the present invention, the at least one H—₃O⁺ ion donor is selected from the group consisting of hydrochloric acid, sulphuric acid, sulphurous acid, phosphoric acid, citric acid, oxalic acid, acetic acid, formic acid, and mixtures thereof. Preferably the at least one H₃O⁺ ion donor is selected from the group consisting of hydrochloric acid, sulphuric acid, sulphurous acid, phosphoric acid, oxalic acid, H₂PO₄⁻, being at least partially neutralised by a corresponding cation such as Li⁺, Na⁺ or K⁺, HPO₄²⁻, being at least partially neutralised by a corresponding cation such as Li⁺, Nat, K⁺, Mg²⁺, or Ca²⁺ and mixtures thereof, more preferably the at least one acid is selected from the group consisting of hydrochloric acid, sulphuric acid, sulphurous acid, phosphoric acid, oxalic acid, or mixtures thereof, and most preferably, the at least one H₃O⁺ ion donor is phosphoric acid.

The one or more H₃O⁺ ion donor can be added to the suspension as a concentrated solution or a more diluted solution. Preferably, the molar ratio of the H—₃O⁺ ion donor to the natural or precipitated calcium carbonate is from 0.01 to 4, more preferably from 0.02 to 2, even more preferably 0.05 to 1 and most preferably 0.1 to 0.58.

As an alternative, it is also possible to add the H₃O⁺ ion donor to the water before the natural or precipitated calcium carbonate is suspended.

In a next step, the natural or precipitated calcium carbonate is treated with carbon dioxide. If a strong acid such as sulphuric acid or hydrochloric acid is used for the H₃O⁺ ion donor treatment of the natural or precipitated calcium carbonate, the carbon dioxide is automatically formed. Alternatively or additionally, the carbon dioxide can be supplied from an external source.

H₃O⁺ ion donor treatment and treatment with carbon dioxide can be carried out simultaneously which is the case when a strong or medium-strong acid is used. It is also possible to carry out H₃O⁺ ion donor treatment first, e.g. with a medium strong acid having a pKₐ in the range of 0 to 2.5 at 20° C., wherein carbon dioxide is formed in situ, and thus, the carbon dioxide treatment will automatically be carried out simultaneously with the H₃O⁺ ion donor treatment, followed by the additional treatment with carbon dioxide supplied from an external source.

In a preferred embodiment, the H₃O⁺ ion donor treatment step and/or the carbon dioxide treatment step are repeated at least once, more preferably several times. According to one embodiment, the at least one H₃O⁺ ion donor is added over a time period of at least about 5 min, preferably at least about 10 min, typically from about 10 to about 20 min, more preferably about 30 min, even more preferably about 45 min, and sometimes about 1 h or more.

Subsequent to the H₃O⁺ ion donor treatment and carbon dioxide treatment, the pH of the aqueous suspension, measured at 20° C., naturally reaches a value of greater than 6.0, preferably greater than 6.5, more preferably greater than 7.0, even more preferably greater than 7.5, thereby preparing the surface-reacted natural or precipitated calcium carbonate as an aqueous suspension having a pH of greater than 6.0, preferably greater than 6.5, more preferably greater than 7.0, even more preferably greater than 7.5.

Further details about the preparation of the surface-reacted natural calcium carbonate are disclosed in WO0039222 A1, WO2004083316 A1, WO2005121257 A2, WO2009074492 A1, EP2264108 A1, EP2264109 A1 and US20040020410 A1, the content of these references herewith being included in the present application.

Similarly, surface-reacted precipitated calcium carbonate is obtained. As can be taken in detail from WO2009074492 A1, surface-reacted precipitated calcium carbonate is obtained by contacting precipitated calcium carbonate with H₃O⁺ ions and with anions being solubilized in an aqueous medium and being capable of forming water-insoluble calcium salts, in an aqueous medium to form a slurry of surface-reacted precipitated calcium carbonate, wherein said surface-reacted precipitated calcium carbonate comprises an insoluble, at least partially crystalline calcium salt of said anion formed on the surface of at least part of the precipitated calcium carbonate.

Said solubilized calcium ions correspond to an excess of solubilized calcium ions relative to the solubilized calcium ions naturally generated on dissolution of precipitated calcium carbonate by H₃O⁺ ions, where said H₃O⁺ ions are provided solely in the form of a counterion to the anion, i.e. via the addition of the anion in the form of an acid or non-calcium acid salt, and in absence of any further calcium ion or calcium ion generating source.

Said excess solubilized calcium ions are preferably provided by the addition of a soluble neutral or acid calcium salt, or by the addition of an acid or a neutral or acid non-calcium salt which generates a soluble neutral or acid calcium salt in situ.

Said H₃O⁺ ions may be provided by the addition of an acid or an acid salt of said anion, or the addition of an acid or an acid salt which simultaneously serves to provide all or part of said excess solubilized calcium ions.

In a further preferred embodiment of the preparation of the surface-reacted natural or precipitated calcium carbonate, the natural or precipitated calcium carbonate is reacted with the one or more H₃O⁺ ion donors and/or the carbon dioxide in the presence of at least one compound selected from the group consisting of silicate, silica, aluminium hydroxide, earth alkali aluminate such as sodium or potassium aluminate, magnesium oxide, or mixtures thereof. Preferably, the at least one silicate is selected from an aluminium silicate, a calcium silicate, or an earth alkali metal silicate. These components can be added to an aqueous suspension comprising the natural or precipitated calcium carbonate before adding the one or more H₃O⁺ ion donors and/or carbon dioxide.

Alternatively, the silicate and/or silica and/or aluminium hydroxide and/or earth alkali aluminate and/or magnesium oxide component(s) can be added to the aqueous suspension of natural or precipitated calcium carbonate while the reaction of natural or precipitated calcium carbonate with the one or more H₃O⁺ ion donors and carbon dioxide has already started. Further details about the preparation of the surface-reacted natural or precipitated calcium carbonate in the presence of at least one silicate and/or silica and/or aluminium hydroxide and/or earth alkali aluminate component(s) are disclosed in WO2004083316 A1, the content of this reference herewith being included in the present application.

The surface-reacted calcium carbonate can be kept in suspension, optionally further stabilised by a dispersant. Conventional dispersants known to the skilled person can be used. A preferred dispersant is comprised of polyacrylic acids and/or carboxymethylcelluloses.

Alternatively, the aqueous suspension described above can be dried, thereby obtaining the solid (i.e. dry or containing as little water that it is not in a fluid form) surface-reacted natural or precipitated calcium carbonate in the form of granules or a powder.

The surface reacted calcium carbonate may have different particle shapes, such as e.g. the shape of roses, golf balls and/or brains.

According to one embodiment of the present invention the mineral material is surface-reacted calcium carbonate and/or a mixture of surface-reacted calcium carbonate and hydromagnesite, and the natural ground calcium carbonate is selected from the group consisting of marble, chalk, limestone, and mixtures thereof, or the precipitated calcium carbonate is selected from the group consisting of precipitated calcium carbonates having an aragonitic, vateritic or calcitic crystal form, and mixtures thereof.

According to a further embodiment the mineral is surface-reacted calcium carbonate and/or a mixture of surface-reacted calcium carbonate and hydromagnesite, and the at least one H₃O⁺ ion donor is selected from the group consisting of hydrochloric acid, sulphuric acid, sulphurous acid, phosphoric acid, citric acid, oxalic acid, an acidic salt, acetic acid, formic acid, and mixtures thereof, preferably the at least one H₃O⁺ ion donor is selected from the group consisting of hydrochloric acid, sulphuric acid, sulphurous acid, phosphoric acid, oxalic acid, H₂PO₄⁻, being at least partially neutralised by a cation selected from Li⁺, Na⁺ and/or K⁺, HPO₄²⁻, being at least partially neutralised by a cation selected from Li⁺, Na⁺, K⁺, Mg²⁺, and/or Ca²⁺, and mixtures thereof, more preferably the at least one H₃O⁺ ion donor is selected from the group consisting of hydrochloric acid, sulphuric acid, sulphurous acid, phosphoric acid, oxalic acid, or mixtures thereof, and most preferably, the at least one H₃O⁺ ion donor is phosphoric acid.

According to one embodiment of the present invention, the surface-reacted calcium carbonate comprises an water-insoluble, at least partially crystalline calcium salt of an anion of the at least one acid, which is formed on the surface of the natural ground calcium carbonate or precipitated calcium carbonate. According to one embodiment, the water-insoluble, at least partially crystalline salt of an anion of the at least one acid covers the surface of the natural ground calcium carbonate or precipitated calcium carbonate at least partially, preferably completely. Depending on the employed at least one acid, the anion may be sulphate, sulphite, phosphate, citrate, oxalate, acetate, formiate and/or chloride.

According to one embodiment, use of a mineral material as anti-pollution cosmetic agent is provided, wherein the mineral material has
a volume median particle size d₅₀(vol) from 0.1 to 90 μm,
a volume top cut particle size d₉₈(vol) of below 250 μm, and
is selected from surface-reacted calcium carbonate, or a mixture of surface-reacted calcium carbonate and hydromagnesite,
wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

### Hydromagnesite

According to one embodiment of the present invention, the mineral material is hydromagnesite and/or a mixture of surface-reacted calcium carbonate as defined herein and hydromagnesite. According to another embodiment of the present invention, the mineral material is hydromagnesite. According to still another embodiment of the present invention, the mineral material is a mixture of surface-reacted calcium carbonate as defined herein and hydromagnesite.

Hydromagnesite or basic magnesium carbonate, which is the standard industrial name for hydromagnesite, is a naturally occurring mineral which is found in magnesium rich minerals such as serpentine and altered magnesium rich igneous rocks, but also as an alteration product of brucite in periclase marbles. Hydromagnesite is described as having the following formula Mg₅(CO₃)₄(OH)₂·4H₂O.

It should be appreciated that hydromagnesite is a very specific mineral form of magnesium carbonate and occurs naturally as small needle-like crystals or crusts of acicular or bladed crystals. In addition thereto, it should be noted that hydromagnesite is a distinct and unique form of magnesium carbonate and is chemically, physically and structurally different from other forms of magnesium carbonate. Hydromagnesite can readily be distinguished from other magnesium carbonates by x-ray diffraction analysis, thermogravimetric analysis or elemental analysis. Unless specifically described as hydromagnesite, all other forms of magnesium carbonates (e.g. artinite (Mg₂(CO₃)(OH)₂·3H₂O), dypingite (Mg₅(CO₃)₄(OH)₂·5H₂O), giorgiosite (Mg₅(CO₃)₄(OH)₂·5H₂O), pokrovskite (Mg₂(CO₃)(OH)₂·0.5H₂O), magnesite (MgCO₃), barringtonite (MgCO₃·2H₂O), lansfordite (MgCO₃·5H₂O) and nesquehonite (MgCO₃·3H₂O)) are not hydromagnesite within the meaning of the present invention and do not correspond chemically to the formula described above.

Besides the natural hydromagnesite, precipitated hydromagnesite (or synthetic magnesium carbonate) can be prepared. For instance, U.S. Pat. No. 1,361,324 A, U.S. Pat. No. 935,418 A, GB548197 A, and GB544907 A generally describe the formation of aqueous solutions of magnesium bicarbonate (typically described as “Mg(HCO₃)₂”), which is then transformed by the action of a base, e.g., magnesium hydroxide, to form hydromagnesite. Other processes described in the art suggest to prepare compositions containing both, hydromagnesite and magnesium hydroxide, wherein magnesium hydroxide is mixed with water to form a suspension which is further contacted with carbon dioxide and an aqueous basic solution to form the corresponding mixture (see, e.g., U.S. Pat. No. 5,979,461 A).

According to one embodiment of the present invention, the hydromagnesite is natural hydromagnesite and/or precipitated hydromagnesite, and preferably precipitated hydromagnesite.

It is appreciated that the hydromagnesite can be one or a mixture of different types of hydromagnesite. In one embodiment of the present invention, the hydromagnesite comprises, preferably consists of, one type of precipitated hydromagnesite. Alternatively, the hydromagnesite comprises, preferably consists of, two or more types of hydromagnesites. For example, the hydromagnesite comprises, preferably consists of, two or three kinds of hydromagnesites. Preferably, the precipitated hydromagnesite comprises, more preferably consists of, one kind of hydromagnesite.

According to one embodiment, use of a mineral material as anti-pollution cosmetic agent is provided, wherein the mineral material has
a volume median particle size d₅₀(vol) from 0.1 to 90 μm,
a volume top cut particle size d₉₈(vol) of below 250 μm, and
is selected from hydromagnesite, or a mixture of hydromagnesite and surface-reacted calcium carbonate,
wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

### Cosmetic Anti-Pollution Composition

The present invention relates to the use of a mineral material as defined herein as anti-pollution cosmetic agent. According to one embodiment of the present invention, the mineral material is used in a cosmetic composition. Thus, according to one embodiment, use of a mineral as anti-pollution cosmetic agent in a cosmetic composition is provided, wherein the mineral material has a volume median particle size d₅₀(vol) from 0.1 to 90 μm, a volume top cut particle size d₉₈(vol) of below 250 μm, and is selected from surface-reacted calcium carbonate, hydromagnesite, or mixtures thereof, wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

According to a further aspect of the present invention, an anti-pollution cosmetic composition comprising a mineral material is provided, wherein the mineral material has
a volume median particle size d₅₀(vol) from 0.1 to 90 μm,
a volume top cut particle size d₉₈(vol) of below 250 μm, and
is selected from surface-reacted calcium carbonate, hydromagnesite, or mixtures thereof,
   wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

According to one embodiment, an anti-pollution cosmetic composition comprising a mineral material is provided, wherein the mineral material has
a volume median particle size d₅₀(vol) from 0.1 to 90 μm,
a volume top cut particle size d₉₈(vol) of below 250 μm, and
is selected from hydromagnesite, or a mixture of hydromagnesite and surface-reacted calcium carbonate,
wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

According to another embodiment, an anti-pollution cosmetic composition comprising a mineral material is provided, wherein the mineral material has
a volume median particle size d₅₀(vol) from 0.1 to 90 μm,
a volume top cut particle size d₉₈(vol) of below 250 μm, and
is selected from surface-reacted calcium carbonate, or a mixture of surface-reacted calcium carbonate and hydromagnesite,
wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

It was surprisingly found by the inventors that the use of the mineral material as defined herein in a cosmetic composition can protect keratin material such as skin, nails, or hair from pollutants.

For example, it was found that the use of the mineral material as defined herein as anti-pollution cosmetic agent in a cosmetic composition can improve the cleansing capacity of the formulation and may reduce the quantity of polluting agents deposited on the skin after rinsing.

The inventors of the present invention also found that in addition to the protective effect against pollutants, the anti-pollution cosmetic agent may improve the skin caring effectivity of a cosmetic composition. Without wishing to be bound by theory, it is believed that the mineral material as defined herein may have the capability to mineralize the skin, especially in form of calcium ions. Such mineralization may induce the biosynthesis of collagen fibres, elastins and/or glycosaminoglycans, and therefore a strengthening of the extracellular matrix of the epidermis and/or dermis.

Moreover, it was found that the sensory properties of a cosmetic composition can be improved by including the anti-pollutant cosmetic agent according to the present invention. For example, it was found that the inventive cosmetic composition is less greasy and sticky compared to a composition having the same ingredients except for the mineral material as defined herein. Moreover, the inventive composition can spread more easily, form a more uniform film and dry faster, when applied to the skin, hair, or nails.

It is appreciated that the amount of the mineral material in the anti-pollution cosmetic composition may vary in a wide range and may be dependent on the cosmetic composition to be prepared and/or the manufacturer's needs and/or legal requirements. For example, in case a cosmetic composition in form of e.g. a paste or an emulsion is prepared, the amount of the mineral material may be below 50 wt.-%, based on the total weight of the cosmetic composition. On the other hand, in case a cosmetic composition in form of e.g. a powder is prepared, the amount of mineral material may be above 50 wt.-%, based on the total weight of the cosmetic composition.

In general, the mineral material can thus be present in the cosmetic composition in an amount from 0.1 to 90 wt.-%, based on the total weight of the cosmetic composition, and preferably from 0.5 to 80 wt.-%.

According to one embodiment of the present invention, the mineral material is present in the cosmetic composition in an amount from 0.1 to 50 wt.-%, based on the total weight of the cosmetic composition, preferably from 0.5 to 20 wt.-%, more preferably from 1 to 10 wt.-%, and most preferably from 3 to 6 wt.-%.

In an alternative embodiment of the present invention, the mineral material is present in the cosmetic composition in an amount from 50 to 90 wt.-%, based on the total weight of the cosmetic composition, and preferably from 60 to 80 wt.-%.

In case the cosmetic composition is prepared in form of a paste or an emulsion, i.e. not in form of a powder, the pH value of the composition may be adjusted to any value suitable for a cosmetic composition. Thus, the cosmetic composition as described herein is not limited to a specific pH value.

The inventors surprisingly found that the pH value of the cosmetic composition according to the invention can be adjusted to a pH value of $7.5, and can even be adjusted to a pH value from 4.0 to 7.0 without showing a negative impact on the stability of the mineral material particles. Usually cosmetic compositions containing, for example, ground calcium carbonate tend to become unstable when the pH value is adjusted below 7.05, and especially below 7.0, due to the liberation of carbon dioxide from the carbonate in the acidic medium. Thus, the inventive cosmetic composition has an improved acid resistance compared to prior art cosmetic products containing, for example, conventional ground calcium. This is particularly advantageous since cosmetic products are usually formulated to have a preferred pH value of below 7.5, or of below 7.0 in order to approach or match the natural PH level of the skin.

The cosmetic composition is, however, not limited to a pH value of ≤7.5, and may also be adjusted to a pH value of $8.5. According to one embodiment, the cosmetic composition has a pH value of $8.5, preferably ≤8.0, more preferably ≤ 7.5, even more preferably ≤7.0, and most preferably from 4.0 to 7.0.

The cosmetic composition may further comprise water and/or at least one oil. Thus, according to one embodiment of the present invention, the cosmetic composition further comprises water. According to another embodiment, the cosmetic composition further comprises at least one oil. According to a preferred embodiment, the cosmetic composition further comprises water and at least one oil. An “oil” in the meaning of the present invention is a liquid or solid silicon- and/or hydrocarbon-containing compound.

The water may be selected from tap water, distilled water, deionized water, or mixtures thereof, and preferably is deionized water.

The at least one oil may be selected from any oil which is suitable to be used in cosmetic and/or skin care compositions. Oils which are suitable for use in cosmetic and/or skin care compositions are known to the skilled person and are described in, for example, Regulation EC No 1223/2009 of the European Parliament and of the Council of 30 Nov. 2009, and must not form part of the list of prohibited substances disclosed therein.

According to one embodiment of the present invention, the at least one oil is selected from the group consisting of vegetable oils and esters thereof, alkanecoconutester, plant extracts, animal fats, siloxanes, silicones, fatty acids and esters thereof, petrolatum, glycerides and pegylated derivatives thereof, and mixtures thereof.

For example, a suitable vegetable oil may be palm oil, soybean oil, rapeseed oil, sunflower seed oil, peanut oil, cottonseed oil, palm kernel oil, coconut oil, olive oil, jojoba oil, corn oil, jumbú oil, guava oil, grape seed oi, hazelnut oil, linseed oil, rice bran oil, safflower oil, sesame oil, acai palm oil, graviola oil, tucuma oil, brazil oil, carapa oil, buriti oil, passion fruit oil or pracaxi oil.

Suitable plant extracts may be prepared, for example, from Castanea sativa, Prunus dulcis, Juglans regia L., Olea europaea, Helichrysum stoechas, Quercus robur, Glycyrrhiza glabra, Vitis vinifera, Crataegus monogyna Jacq, or Pinus Pinaster.

Suitable animal fats can be obtained, for example, from tallow.

Suitable siloxanes are, for example, dimethicone, cetyl dimethicone, dimethiconol, detearyl methicone, cyclopentasiloxane, cyclomethicone, stearyl dimethicone, trimethylsilylamodimethicone, stearoxy dimethicone, amodimethicone, behenoxy dimethicone, dimethicone copolyol, polysiloxane, laurylmethicone copolyol or cetyl dimethicone copolyol.

Suitable fatty acids are, for example, palmitic acid, stearic acid, myristic acid, oleic acid, palmitoleic acid, linoleic acid, linolenic acid, capric acid, caprylic acid, arachidonic acid and esters thereof.

Suitable petrolatum may be any petrolatum with a refined grade approved for cosmetic use, and preferably has a melting point between 35° C. and 70° C.

Suitable glycerides are, for example, mono-, di, or triglycerides from palmitic acid, stearic acid, myristic acid, oleic acid, palmitoleic acid, linoleic acid, linolenic acid, capric acid, caprylic acid, and mixtures thereof.

In one embodiment, the at least one oil comprises, preferably consists of, one oil. Alternatively, the at least one oil comprises, preferably consists of, two or more oils. For example, the at least one oil comprises, preferably consists of, two or three oils. Preferably, the at least one oil comprises, preferably consists of, two or more oils.

It is appreciated that the cosmetic composition may comprise the water and/or the at least one oil and their amounts in dependence of the cosmetic composition to be prepared and/or the manufacturer's needs. According to one embodiment, the water is present in an amount of from 1 to 95 wt.-%, preferably from 15 to 90 wt.-%, more preferably from 25 to 80 wt.-%, even more preferably from 35 to 75 wt.-%, and most preferably from 45 to 65 wt.-%, based on the total weight of the cosmetic composition. According to another embodiment, the at least one oil is present in an amount of from 1 to 95 wt.-%, preferably from 2 to 75 wt.-%, more preferably from 5 to 55 wt.-%, even more preferably from 7.5 to 35 wt.-%, and most preferably from 10 to 20 wt.-%, based on the total weight of the cosmetic composition.

In case the cosmetic composition comprises water and at least one oil, the composition may be a water-based dispersion or an oil-based dispersion. Thus, according to one embodiment, the cosmetic composition is a water-based dispersion. According to another embodiment, the composition is an oil-based dispersion. According to a preferred embodiment, the cosmetic composition is a water-based dispersion. A “water-based dispersion” in the meaning of the present invention refers to a composition wherein water forms a continuous phase and the oil a dispersed phase, i.e. the oil is dispersed in the continuous water phase. An “oil-based dispersion” in the meaning of the present invention refers to a composition wherein oil forms a continuous phase and water a dispersed phase, i.e. water is dispersed in the continuous water phase. According to yet another embodiment, the water is present in an amount of from 1 to 95 wt.-%, preferably from 15 to 90 wt.-%, more preferably from 25 to 80 wt.-%, even more preferably from 35 to 75 wt.-%, and most preferably from 45 to 65 wt.-%, and the at least one oil is present in an amount of from 1 to 95 wt.-%, preferably from 2 to 75 wt.-%, more preferably from 5 to 55 wt.-%, even more preferably from 7.5 to 35 wt.-%, and most preferably from 10 to 20 wt.-%, based on the total weight of the cosmetic composition.

As described above, the intra and interpore structure of the mineral material can make it a superior agent to deliver previously adsorbed and/or absorbed materials over time relative to common materials having similar specific surface areas. Thus, generally, any agent fitting into the intra- and/or inter particle pores of the mineral material is suitable to be transported by the mineral material defined herein. Accordingly, it is possible that the cosmetic composition comprises at least one active agent being adsorbed onto and/or absorbed into the surface of the mineral material. According to one embodiment of the present invention, the cosmetic composition comprises at least one active agent being adsorbed onto and/or absorbed into the surface of the mineral material.

The cosmetic composition may also comprise further additives. Additives that are suitable for cosmetic compositions are known to the skilled person and are described in, for example, Regulation EC No 1223/2009 of the European Parliament and of the Council of 30 Nov. 2009, and must not form part of the list of prohibited substances disclosed therein. According to one embodiment of the present invention, the cosmetic composition further comprises at least one additive selected from the group consisting of bleaching agents, thickeners, stabilizers, chelating agents, preserving agents, wetting agents, emulsifiers, emollients, fragrances, colorants, skin tanning compounds, antioxidants, minerals, pigments, UV-A and/or UV-B filter, and mixtures thereof.

For example, the emulsifier can be an ionic emulsifier, more preferably and anionic or cationic emulsifier. The emulsifier can be of natural vegetable origin e.g. polyglycerol ester or synthetic. More preferably, the emulsifier may be selected from the group comprising PEG compounds, PEG-free emulsifier, silicone-based emulsifier, silicones, waxes and mixtures thereof. For example, the emulsifier may be selected from the group comprising PEG compounds such as PEG-8 myristate, PEG-30 glyceryl cocoate, PEG-80 glyceryl cocoate, PEG-15 soyamide/IPDI copolymer, PEG-40 sorbitan peroleate, PEG-150 stearate and mixtures thereof, carbomer, carboxymethylcellulose, ceresin (aka mineral wax), diethanolamine (DEA), isopropyl stearate, isopropyl laurate, isopropyl palmitate, isopropyl oleate, polysorbate 20, polysorbate 60, polysorbate 80, propylene glycol, sorbitan stearate, sorbitan laurate, sorbitan palmitate, sorbitan oleate, steareth-20, triethanolamine (TEA), beeswax, candelilla wax, carnauba wax, cetearyl alcohol, cetearyl wheat bran glycosides, cetearyl wheat straw glycosides, decyl glucoside, jojoba, lecithin, vegetable glycerin, xanthan gum, coco glucoside, coconut alcohol, arachidyl alcohol, behenyl alcohol, arachidyl glucoside, and mixtures thereof.

The fragrance may be selected from a natural and/or synthetic fragrance known as being suitable in cosmetic formulations.

The colorant may be selected from a natural and/or synthetic colorant, pigment or dye such as Fe₂O₃, ZnO, TiO₂, mica, talc, bismuth oxychloride, and mixtures thereof.

According to one embodiment, the skin tanning compound is preferably dihydroxyacetone (DHA) and/or erythrulose. For example, the skin tanning compound may be dihydroxyacetone (DHA) or erythrulose. Alternatively, the skin tanning compound may be dihydroxyacetone (DHA) in combination with erythrulose.

According to one embodiment, the cosmetic composition further comprises at least one emollient. Examples of suitable emollients are isocetylstearoylstearate, ethylhexyl stearate, octyldodecyl stearoyl stearate, isocetyl stearate, isopropyl isostearate, isostearyl isostearate, ethylhexyl hydroxystearate, ethylhexyl palmitate, isopropyl palmitate, neopentyl glycol diheptanoate, ethylhexyl isononanoate, isononyl isononanoate, cetearyl isononanoate, cetearyl octanoate, diisopropyl adipate, dicapryl adipate, diisostearylmalate, decyl oleate, isodecyl oleate, diisopropyl myristate, isostearyl neopentanoate, octyl dodecyl neopentanoate, ethylhexyl cocoate, PEG-7 glyceril cocoate, C12-15 alkyl benzoate, C16-17 alkyl benzoate, stearyl benzoate, isostearyl benzoate, ethylhexyl benzoate, octyldodecyl benzoate, cocoglyceride, coconut alkanes, coco-caprylate/caprate, and mixtures thereof. For example, the cosmetic composition may further comprise a mixture of cocoglyceride, isononyl isononanoate, coconut alkanes and coco-caprylate/caprate as emollient.

Additionally or alternatively, the cosmetic composition further comprises at least one thickener. Examples of suitable thickener for a water-based dispersion are thickener based on silicate such as magnesium silicate, aluminium silicate and mixtures thereof, hydroxyethylcellulose, cellulose, microcrystalline cellulose, xanthan gum or polyacrylamide. Examples of suitable thickener for an oil-based dispersion are selected from the group comprising silicate such as magnesium silicate, aluminium silicate, silica dimethylsilicate, hydrophobic fumed silica, polyacrylic acid, salts of polyacrylic acid, derivatives of polyacrylic acid, PEG compounds such as PEG-8 myristate, PEG-30 glyceryl cocoate, PEG-80 glyceryl cocoate, PEG-15 soyamide/IPDI copolymer, PEG-40 sorbitan peroleate, PEG-150 stearate and mixtures thereof, methyl cellulose, ethyl cellulose, propyl cellulose, carboxymethylcellulose, xanthan gum, ammonium acryloyldimethyltaurate/VP copolymer and mixtures thereof.

Additionally or alternatively, the cosmetic composition further comprises at least one preserving agent. Examples of suitable preserving agents are phenoxyethanol, ethylhexylglycerin, parabens such as methyl paraben, ethyl paraben, propyl paraben, butyl paraben, isobutyl paraben and mixtures thereof, benzoic acid, sodium benzoate, sorbic acid, potassium sorbate and mixtures thereof, or plant extracts with preservative function such as rosemary extracts. For example, said mixture may comprise phenoxyethanol, methyl paraben, ethyl paraben and isobutyl paraben.

Examples of suitable chelating agents are a polyphosphate, ethylenediamine-N,N,N′,N′-tetraacetic acid (EDTA), pyridine-2,6-dicarboxylic acid (DPA), diethylenetriaminepentaacetic acid (DTPA), N,N-bis(carboxymethyl)glycine (NTA), ammonium diethyldithiophosphate (DDPA), disodium ethylenediamine-tetraacetate (Na₂H₂EDTA), calcium-disodium-ethylenediamine-tetraacetate (CaNa₂EDTA), citric acid and salts of citric acid, sodium gluconate, and mixtures thereof.

Examples of suitable wetting agents are primary alcohols such as 1-ethanol, 1-propanol, 1-butanol, isobutanol 1-pentanol, isoamyl alcohol, 2-methyl-1butanol, 1-hexanol, 1-heptanol, 1-octanol, 1-nonanol, 1-decanol, 1-undecanol, 1-dodecanol, 1-tridecanol, 1-tetradecanol, 1-pentadecanol, cetyl alcohol, 1-heptadecanol, stearyl alcohol, 1-nonadecanol and mixtures thereof, secondary alcohols such as isopropanol, 2-butanol, 2-pentanol, 2-hexanol, 2-heptanol and mixtures thereof, tertiary alcohols such as tert.-butyl alcohol, tert.-amyl alcohol, 2-methyl-2-pentanol, 2-methylhexan-2-ol, 2-methylheptan-2-ol, 3-methyl-3-pentanol, 3-methyloctan-3-ol and mixtures thereof, diols such as 1,2-diols or 1,3-diols, e.g. 1,3-propandiol, urea, and mixtures thereof.

Examples of suitable antioxidants are butylhydroxyanisol (BHA), butylhydroxytoluol (BHT), gallate, carotinoid, polyphenols such as resveratrol, flavonoid and mixtures thereof, derivatives of polyphenols, ascorbic acid and salts thereof, tocopherol and salts thereof, betacarotin, ubichinon, tocotrienol, dihydroquercetin, antioxidants of natural origin, and mixtures thereof.

Examples of suitable pigments are inorganic red pigments such as iron oxide, ferric hydroxide and iron titanate, inorganic brown pigments such as γ-iron oxide, inorganic yellow pigments such as yellow iron oxide and yellow ocher, inorganic black pigments such as black iron oxide and carbon black, inorganic purple pigments such as manganese violet and cobalt violet, inorganic green pigments such as chromium hydroxide, chrome oxide, cobalt oxide and cobalt titanate, inorganic blue pigments such as iron blue and ultramarine, particulate powders such as particulate titanium oxide, particulate cerium oxide and particulate zinc oxide, laked tar dyes, laked natural dyes, and synthetic resin powders combining foregoing powders.

The bleaching agent may be selected from one or more of a vitamin B3 compound or its derivative e.g. niacin, nicotinic acid or niacinamide or other well-known bleaching agents e.g. adapalene, aloe extract, ammonium lactate, anethole derivatives, apple extract, arbutin, azelaic acid, kojic acid, bamboo extract, bearberry extract, bletilla tuber, bupleurum falcatum extract, burnet extract, butyl hydroxy anisole, butyl hydroxy toluene, citrate esters, Chuanxiong, Dang-Gui, deoxyarbutin, 1,3-diphenyl propane derivatives, 2,5-dihydroxybenzoic acid and its derivatives, 2-(4-acetoxyphenyl)-1,3-dithane, 2-(4-hydroxyphenyl)-1,3-dithane, ellagic acid, escinol, estragole derivatives, Fadeout (Pentapharm), Fangfeng, fennel extract, ganoderma extract, gaoben, Gatuline Whitening (Gattlefosse), genistic acid and its derivatives, glabridin and its derivatives, gluco pyranosyl-1-ascorbate, gluconic acid, glycolic acid, green tea extract, 4-hydroxy-5-methyl-3[2H]-furanone, hydroquinone, 4-hydroxyanisole and its derivatives, 4-hydroxy benzoic acid derivatives, hydroxycaprylic acid, inositol ascorbate, lemon extract, linoleic acid, magnesium ascorbyl phosphate, Melawhite (Pentapharm), morus alba extract, mulberry root extract, 5-octanoyl salicylic acid, parsley extract, phellinus linteus extract, pyrogallol derivatives, 2,4-resorcinol derivatives, 3,5-resorcinol derivatives, rose fruit extract, salicylic acid, Song-Yi extract, 3,4,5-trihydroxybenzyl derivatives, tranexamic acid, vitamins like vitamin B6, vitamin B12, vitamin C, vitamin A, dicarboxylic acids, resorcinol derivatives, extracts from plants viz. rubia and symplocos, hydroxycarboxylic acids like lactic acid and their salts e.g. sodium lactate, and mixtures thereof. Vitamin B3 compound or its derivative e.g. niacin, nicotinic acid or niacinamide are the more preferred bleaching agents, most preferred being niacinamide. Niacinamide, when used, is preferably present in an amount in the range of 0.1 to 10 wt.-%, more preferably 0.2 to 5 wt.-%, based on the total weight of the cosmetic composition.

The minerals may be selected from any minerals suitable for the use in a cosmetic composition. For example, the cosmetic composition may contain silicates such as talc, mica and/or kaolin.

UV-A and/or UV-B filter may be selected from inorganic UV filter and/or organic UV filter. Suitable inorganic UV filter are, for example, selected from the group consisting of titanium dioxide, zinc oxide, iron oxide, hydroxyapatite, cerium oxide, calcium-doped cerium oxide, cerium phosphate, and mixtures thereof. Suitable organic UV filter are, for example, selected from the group comprising cinnamic acid and its salts, derivatives of salicylic acid and its salts, benzophenones, derivatives of aminobenzoic acid and its salts, dibenzoylmethanes, benzylidenecamphor derivatives, benzimidazole derivatives, diphenylacrylate derivatives, acrylamide derivatives, benzotriazole derivatives, triazine derivatives, benzalmalonate derivatives, aminobenzoate derivatives, octocrylene, and mixtures thereof.

It is appreciated that the cosmetic composition may comprise the at least one further additive and its amount in dependence of the cosmetic composition to be prepared and/or the manufacturer's needs. For example, the cosmetic composition may comprise 0.1 to 10 wt.-% of thickeners, stabilizers, chelating agents, bleaching agents, wetting agents, emulsifiers, emollients, and/or skin tanning compounds, and/or 0.1 to 15 wt.-% of preserving agents, fragrances, colorants, antioxidants, minerals, pigments, UV-A and/or UV-B filter wherein the wt.-% is based on the total weight of the cosmetic composition.

In one embodiment, the at least one additive comprises, preferably consists of, one additive. Alternatively, the at least one additive comprises, preferably consists of, two or more additives. For example, the at least one additive comprises, preferably consists of, ten to fifteen additives. Preferably, the at least one additive comprises, preferably consists of, two or more additives.

The cosmetic composition may be provided in the form of any cosmetic product being applicable to the skin of the face and/or body, nails, or hair. According to one embodiment of the present invention, the anti-pollution cosmetic composition is a sun protection product, an eye make-up product, a facial make-up product, a lip care product, a hair care product, a hair styling product, a hair cleaning product, a nail care product, a hand care product, a hand cleaning product, a skin care product, a skin cleaning product, a scalp care product, a scalp cleaning product, a facial cleaning product, a make-up remover, a facial mist, a cleaning wipe, an exfoliating product, or a combination product thereof.

Examples of a sun protection product are a sun protection cream, a sun protection lotion, a sun blocker, a sun protection lip balm, or a sun protection spray. Examples of an eye make-up product are eye shadow, mascara, concealer, eye liner, or eye brow pen. Examples of a facial make-up are foundation, BB cream, loose face powder, compact face powder, cream blush, powder blush, or bronzer. Examples of a lip care product are a lip balm, a lip serum, a lip mask, a lip scrub, a lip moisturizer, a lip oil, or a lip butter.

Examples of a hair care product are a hair conditioner, a leave-in hair conditioner, or a hair mask. Examples of a hair styling product are a voluminizing spray, an anti-frizz serum, a hair mousse, a hair gel, or a hair spray. Examples of a hair cleaning product are a liquid shampoo, a dry shampoo, a shampoo bar, a hair cleaning cream, or a hair cleaning gel.

According to one embodiment, the cosmetic composition is a dry shampoo. It was surprisingly found by the inventors of the present invention that a dry shampoo containing a mineral material according to the present invention may remove very effectively sebum from hair and may also provide a significantly increase in hair volume. The dry shampoo may be in form of a powder or in form of a spray-on dry shampoo. Spray-on dry shampoos typically contain one or more propellants, e.g. propane, n-butane, isobutene, dimethyl ether (DME), methyl ethyl ether, nitrous oxide, or carbon dioxide. In addition to the mineral material according to the present invention, the dry shampoo may comprise clay, kaolin, starch, magnesium stearate, alcohol, plant extracts, algae extracts, or mixtures thereof. According to one embodiment, an anti-pollution cosmetic composition comprising a mineral material is provided, wherein the cosmetic composition is a dry shampoo and the mineral material has a volume median particle size d₅₀(vol) from 0.1 to 90 μm, a volume top cut particle size d₉₈(vol) of below 250 μm, and is selected from surface-reacted calcium carbonate, hydromagnesite, or mixtures thereof, wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

Examples of a nail care product are a nail cream, a nail strengthener, or an anti-chew treatener. Examples of a hand care product are hand lotion, hand cream, hand mask, or hand oil. Examples of a hand cleaning product are hand soap, hand scrub, or hand sanitizer. Examples of a skin care product are a skin mask, a skin cream, a skin moisturizer, a skin oil, a skin serum, an anti-wrinkle cream, a skin gel, a body lotion, or a skin tonic. Examples of a skin cleaning product are skin wash, shower lotion, a skin cleansing oil, a skin jelly cleanser, a skin cleanser foam, a face cleansing gel, a face peeling, or a face scrub. Examples of a scalp care product are scalp oil, scalp serum, or scalp mask. Examples of scalp cleaning products are scalp cleaning solution, or scalp scrub.

Examples of a facial cleaning product are a face wash, a face cleansing oil, a face jelly cleanser, a face cleanser foam, a face cleansing gel, a face peeling, or a face scrub. Examples of a make-up remover are make-up remover water, make-up remover gel, make-up remover lotion, make-up remover oil, oil-free make up remover, or make-up remover balm.

Furthermore, the cosmetic composition may have a certain Brookfield viscosity. For the purpose of the present invention, the term “viscosity” or “Brookfield viscosity” refers to Brookfield viscosity. According to one embodiment of the present invention, the cosmetic composition has a Brookfield viscosity in a range from 4 000 to 50 000, preferably from 10 000 to 45 000, more preferably from 15 000 to 40 000, even more preferably from 20 000 to 40 000, and most preferably from 25 000 to 40 000 mPa·s at 25° C.

According to a further aspect of the present invention, a cosmetic method of protecting a keratin material from pollutants is provided, comprising:
(i) providing an anti-pollution cosmetic composition according to the present invention, and
(ii) applying said anti-pollution cosmetic composition onto the keratin material.

According to one embodiments the pollutants are atmospheric pollutants, preferably selected from the group consisting of carbon black, carbon oxides, nitrogen oxides, sulfur oxides, metal oxides, hydrocarbons, organic volatiles, heavy metals, atmospheric particulate matter, fine particulate matter (PM_{2.5}), and mixtures thereof. According to one embodiment the keratin material is skin, nails, and/or hair, preferably keratin material of the human body. The anti-pollution cosmetic composition may be applied onto the keratin material topically, for example, by creaming, dabbing, or spraying.

According to one embodiment, said cosmetic method further comprises the step (iii) of removing the anti-pollution cosmetic composition, for example, by rinsing with water or wiping with a wet cloth.

As noted above, the term “cosmetic” does not encompass a therapeutic application. Thus, a non-therapeutic use of a mineral material as anti-pollution cosmetic agent is provided, wherein the mineral material has a volume median particle size d₅₀(vol) from 0.1 to 90 μm, a volume top cut particle size d₉₈(vol) of below 250 μm, and is selected from surface-reacted calcium carbonate, hydromagnesite, or mixtures thereof, wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

According to a further aspect of the present invention, a non-therapeutic cosmetic method of protecting a keratin material from pollutants is provided, comprising: (i) providing an anti-pollution cosmetic composition according to the present invention, and (ii) applying said anti-pollution cosmetic composition onto the keratin material. According to one embodiment, a mineral material for use in a method of protecting a keratin material from pollutants is provided, comprising: (i) providing an anti-pollution cosmetic composition according to the present invention, and (ii) applying said anti-pollution cosmetic composition onto the keratin material.

### Preparation of the Anti-Pollution Cosmetic Composition

A method for the preparation of the anti-pollution cosmetic composition according to the present invention comprises at least the provision of a mineral material, wherein the mineral material has a volume median particle size d₅₀ from 0.1 to 90 μm, a volume top cut particle size d₉₈ of below 250 μm, and is selected from surface-reacted calcium carbonate, hydromagnesite, or mixtures thereof, wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

The mineral material may be provided in any suitable liquid or dry form. For example, the mineral material may be in form of a powder and/or a suspension. The suspension can be obtained by mixing the mineral material with a solvent, preferably water. The mineral material to be mixed with a solvent, and preferably water, may be provided in any form, for example, as suspension, slurry, dispersion, paste, powder, a moist filter cake or in pressed or granulated form, and preferably is provided as a powder.

The term “dispersion” or “suspension” in the meaning of the present invention refers to a system comprising a dispersing medium or solvent and at least one inorganic particulate material, wherein at least a part of the particles of the at least one inorganic particulate material are present as insoluble solids or suspended particles in the dispersing medium or solvent.

The suspension can be undispersed or dispersed, i.e. the suspension includes a dispersant, and thus, forms a dispersion, e.g. an aqueous dispersion. Suitable dispersants are known in the art, and may be selected, e.g., from polyelectrolytes, polyhydroxystearic acid, acetylacetone, propylamine, oleic acid, polyacrylates, carboxymethylcellulose based dispersants, and mixtures thereof.

The solids content of the suspension, preferably aqueous suspension, of the mineral material may be from 1 to 85 wt.-%, more preferably from 5 to 75 wt.-%, and most preferably from 10 to 40 wt.-%, based on the total weight of the suspension.

In case the mineral material is provided in dry form, the moisture content of the surface-reacted calcium carbonate can be between 0.01 and 5 wt.-%, based on the total weight of the mineral material. The moisture content of the mineral material can be, for example, less than or equal to 1.0 wt.-%, based on the total weight of the mineral material, preferably less than or equal to 0.5 wt.-%, and more preferably less than or equal to 0.2 wt.-%. According to another example, the moisture content of the mineral material may be between 0.01 and 0.15 wt.-%, preferably between 0.02 and 0.10 wt.-%, and more preferably between 0.03 and 0.07 wt.-%, based on the total weight of the mineral material.

The method for the preparation of the cosmetic composition may further comprise the provision of water and/or at least one oil and the mixing of the water and/or at least one oil with the mineral material.

The mixing of the water and/or the at least one oil and the mineral material may be carried out in any manner known by the skilled person. The mixing may be carried out under conventional mixing conditions. The skilled man will adapt these mixing conditions (such as the configuration of mixing pallets and mixing speed) according to his process equipment. It is appreciated that any mixing method which would be suitable to form a cosmetic composition may be used.

In case, the method further comprises the provision of water and at least one oil, the mixing may be carried out in any order. Preferably, the water and the at least one oil are combined and mixed to form a mixture followed by the addition and mixing of the mineral material.

Mixing can be carried out at temperatures typically used for preparing a cosmetic base formulation. Preferably, mixing is carried out at a temperature in the range from 15 to 100° C., more preferably from 20 to 85° C. such as of about 45° C.

The method for the preparation of the cosmetic composition may further comprise the provision of at least one additive. The combining and mixing of the at least one additive and the mineral material may also be carried out under conventional mixing conditions. The skilled man will adapt these mixing conditions (such as the configuration of mixing pallets and mixing speed) according to his process equipment. It is appreciated that any mixing method which would be suitable to form a cosmetic composition may be used.

In case, the method comprises the provision of the mineral material, water and/or at least one oil, and at least one additive, and preferably two or more additives, the combining and mixing may be carried out in any order.

For example, the method for the preparation of the cosmetic composition may comprise the steps of:
a) providing a mineral material as described herein,
b) providing water,
c) providing at least one oil,
d) providing two or more additives,
e) combining and mixing one or more of the two or more additives with water to form a first mixture,
f) combining and mixing one or more of the two or more additives with the at least one oil to form a second mixture
g) combining and mixing the first and the second mixture to form a third mixture,
h) optionally combining and mixing the third mixture with one or more of the two or more additives, to form a fourth mixture,
i) combining and mixing the mineral material with the third mixture of step g) or the fourth mixture of step h).

The scope and interest of the invention will be better understood based on the following examples which are intended to illustrate certain embodiments of the present invention and are non-limitative.

### EXAMPLES

### 1. Measurement Methods

In the following, measurement methods implemented in the examples are described.

### Particle Size Measurement

Volume determined median particle size d₅₀(vol) and the volume determined top cut particle size d₉₈(vol) was evaluated using a Malvern Mastersizer 2000 Laser Diffraction System (Malvern Instruments Plc., Great Britain). The d₅₀(vol) or d₉₈(vol) value indicates a diameter value such that 50% or 98% by volume, respectively, of the particles have a diameter of less than this value. The raw data obtained by the measurement was analyzed using the Mie theory, with a particle refractive index of 1.57 and an absorption index of 0.005. The methods and instruments are known to the skilled person and are commonly used to determine particle size distributions of fillers and pigments. The sample was measured in dry condition without any prior treatment.

The weight determined median particle size d₅₀(wt) and the weight determined top cut particle size d₉₈(wt) was measured by the sedimentation method, which is an analysis of sedimentation behaviour in a gravimetric field. The measurement was made with a Sedigraph™ 5120 of Micromeritics Instrument Corporation, USA. The method and the instrument are known to the skilled person and are commonly used to determine particle size distributions of fillers and pigments. The measurement was carried out in an aqueous solution of 0.1 wt.-% Na₄P₂O₇. The samples were dispersed using a high speed stirrer and supersonicated.

### Specific Surface Area (SSA)

The specific surface area was measured via the BET method according to ISO 9277:2010 using nitrogen and a ASAP 2460 instrument (Micromeritics GmbH, Germany), following conditioning of the sample by heating at 100° C. for a period of 30 minutes. Prior to such measurements, the sample was filtered within a Büchner funnel, rinsed with deionised water and dried at 110° C. in an oven for at least 12 hours.

Intra-Particle Intruded Specific Pore Volume (in cm³/g)

The specific pore volume was measured using a mercury intrusion porosimetry measurement using a Micromeritics Autopore V 9620 mercury porosimeter having a maximum applied pressure of mercury 414 MPa (60 000 psi), equivalent to a Laplace throat diameter of 0.004 μm (˜nm). The equilibration time used at each pressure step was 20 seconds. The sample material was sealed in a 5 cm3 chamber powder penetrometer for analysis. The data were corrected for mercury compression, penetrometer expansion and sample material compression using the software Pore-Comp (Gane, P. A. C., Kettle, J. P., Matthews, G. P. and Ridgway, C. J., “Void Space Structure of Compressible Polymer Spheres and Consolidated Calcium Carbonate Paper-Coating Formulations”, Industrial and Engineering Chemistry Research, 35(5), 1996, 1753-1764).

The total pore volume seen in the cumulative intrusion data can be separated into two regions with the intrusion data from 214 μm down to about 1-4 μm showing the coarse packing of the sample between any agglomerate structures contributing strongly. Below these diameters lies the fine inter-particle packing of the particles themselves. If they also have intra-particle pores, then this region appears bi-modal, and by taking the specific pore volume intruded by mercury into pores finer than the modal turning point, i.e. finer than the bi-modal point of inflection, the specific intra-particle pore volume is defined. The sum of these three regions gives the total overall pore volume of the powder, but depends strongly on the original sample compaction/settling of the powder at the coarse pore end of the distribution.

By taking the first derivative of the cumulative intrusion curve the pore size distributions based on equivalent Laplace diameter, inevitably including pore-shielding, are revealed. The differential curves clearly show the coarse agglomerate pore structure region, the inter-particle pore region and the intra-particle pore region, if present. Knowing the intra-particle pore diameter range it is possible to subtract the remainder inter-particle and inter-agglomerate pore volume from the total pore volume to deliver the desired pore volume of the internal pores alone in terms of the pore volume per unit mass (specific pore volume). The same principle of subtraction, of course, applies for isolating any of the other pore size regions of interest.

### 2. Materials

### 2.1. Mineral Material

### SRCC1

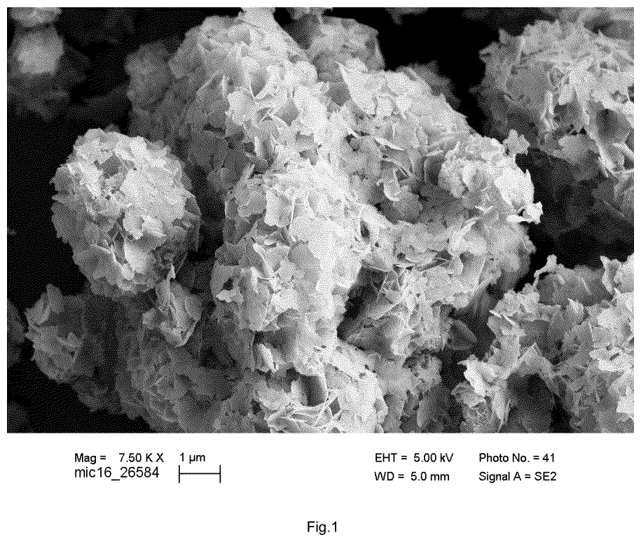

Surface-reacted calcium carbonate, d₅₀(vol)=4.5 μm, d₉₈(vol)=8.6 μm, SSA=96.1 m²/g, intra-particle intruded specific pore volume=1.588 cm3/g (for the pore diameter range of 0.004 to 0.4 μm). A SEM micrograph of SRCC1 is shown in FIG. 1.

### SRCC1 was Prepared According to the Following Procedure:

In a mixing vessel, 10 liters of an aqueous suspension of ground limestone calcium carbonate was prepared by adjusting the solids of a ground limestone calcium carbonate having a weight-determined particle size distribution of 90 wt.-% below 2 μm, based on the total weight of the ground calcium carbonate, such that a solids content of 15 wt.-%, based on the total weight of the aqueous suspension, is obtained.

Whilst mixing the slurry, 2.8 kg phosphoric acid was added in form of an aqueous solution containing 30 wt.-% phosphoric acid to said suspension over a period of 10 minutes. Throughout the whole experiment the temperature of the suspension was maintained at 70° C. After the addition of the acid, the suspension was stirred for additional 5 minutes before removing it from the vessel and drying.

### SRCC2

Surface-reacted calcium carbonate d₅₀(vol)=6.6 μm, d₉₈(vol)=13.7 μm, SSA=59.9 m²/g, intra-particle intruded specific pore volume=0.939 cm3/g (for the pore diameter range of 0.004 to 0.51 μm).

### SRCC2 was Prepared According to the Following Procedure:

In a mixing vessel, 350 litres of an aqueous suspension of ground calcium carbonate was prepared by adjusting the solids content of a ground limestone calcium carbonate having a weight-determined median particle size d₅₀(wt) of 1.3 μm such that a solids content of 10 wt.-%, based on the total weight of the aqueous suspension, is obtained.

Whilst mixing the slurry at a speed of 6.2 m/s, 11.2 kg phosphoric acid was added in form of an aqueous solution containing 30 wt.-% phosphoric acid to said suspension over a period of 20 minutes at a temperature of 70° C. After the addition of the acid, the slurry was stirred for additional 5 minutes, before removing it from the vessel and drying using a jet-dryer.

### PHM

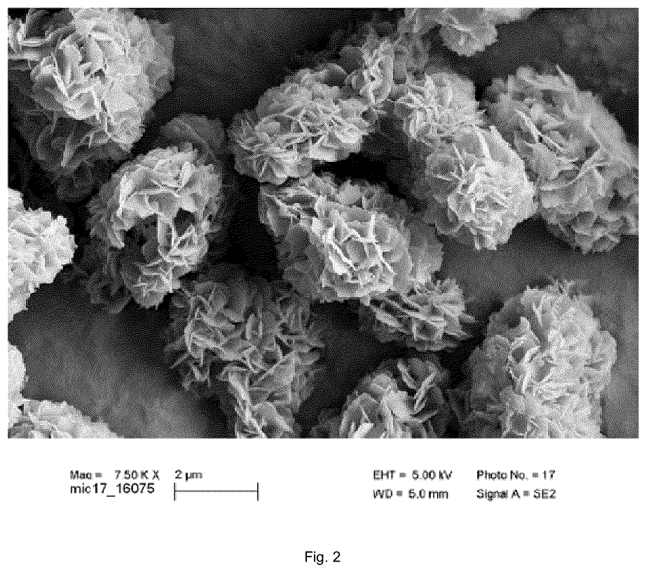

The hydromagnesite was a precipitated hydromagnesite produced by Omya International AG based on published protocols (see e.g. M. Pohl, C. Rainer, M. Esser; Omya Development AG, EP2322581 A1). The hydromagnesite has a d₅₀(vol)=6.17 μm, d₉₈(vol)=27 μm, BET=44.3 m²/g, and an intra-particle intruded specific pore volume=2.008 cm3/g (for the pore diameter range of 0.004 to 0.8 μm). A SEM micrograph of PHM is shown in FIG. 2.

### 2.2. Tested Cosmetic Skin Formulations

In the following the INCI name of the ingredients are used, wherein INCI stands for International Nomenclature of Cosmetic Ingredients.

TABLE 1
Tested cosmetic skin formulations.

| | | | Formulation 1 | Formulation 2 |
|---|---|---|---|---|
| Phase | Ingredients | INCI Nomenclature | (wt.-%) | (wt.-%) |
| A | Lanette O | Cetearyl Alcohol | 2.00 | 2.00 |
| | Imwitor 372P | Glyceryl Stearate Citrate | 5.00 | 5.00 |
| | Almond Oil | Prunus Amygdalus Dulcis | 2.00 | 2.00 |
| | | (Sweet Almond) Oil | | |
| | Apricot Oil | Prunus Armeniaca Kernel Oil | 3.00 | 3.00 |
| | KCC SF1000N -100 cSt | Polydimethylsiloxane | 2.00 | 2.00 |
| | KCC 4130P | Stearyl Dimethicone | 2.00 | 2.00 |
| | Coconut Oil | Cocos nucifera Oil | 3.00 | 3.00 |
| B | Water den. | Aqua (water) | add up to 100 | add up to 100 |
| | 1,2-Propanediol | Propylene Glycol | 4.00 | 4.00 |
| | Glycerin | Glycerin | 3.00 | 3.00 |
| | Xanthan Gum | Xanthan Gum | 0.20 | 0.20 |
| | Potassium Sorbate | Potassium Sorbate | 1.00 | 1.00 |
| | Sodium Chloride | Sodium Chloride | 1.00 | 1.00 |
| C | SRCC1 | Calcium carbonate | 5.00 | |
| | PHM | Hydromagnesite carbonate | — | 5.00 |
| D | Phenonip | Methylparaben (and) | 1.00 | 1.00 |
| | | Ethylparaben (and) Butylparaben | | |
| | | (and) Propylparaben (and) | | |
| | | Phenoxyethanol | | |

| | | |
|---|---|---|
| Total | 100.00 | 100.00 |

The formulations were prepared according to the following protocol:
1. Heat phase A and B separately at 80° C.
2. Add phase B to phase A while stirring
3. Cool down at room temperature
4. Add phase C and D and homogenize
5. Adjust the pH at 6.0 using lactic acid (10%-solution).

### 3. Examples

### 3.1. Cosmetic Skin Formulation

In vivo studies were carried out with the aim to evaluate the protective effect and the cleansing capacity, respectively, against particles (size 1 μm on average) modelling atmospheric pollution after a single standardized application. The study was performed on 21 subjects being between 20 and 45 years old.

The study was carried out on cosmetic products whose safety has been assured by the Sponsor. This study, performed on a cosmetic product coming within the definition of article L. 5131-1 of the French Public Health Code was in accordance with Decree no 2017-884 of May 9, 2017 modifying some regulatory requirements concerning researches involving human subjects. For all these reasons, this study did not require the Ethics Committee Approval and the Competent Authority Authorization.

During the study the subjects had to comply with date and hour of the evaluation visit. They were not allowed to apply any product to test areas the days of the visits to the lab. A shower with the usual product is allowed the day of the evaluation visit to the lab at least 2 hours before measurements.

A protocol deviation was defined as any non-adherence to the final protocol, including wrong inclusion (inclusion criteria or non-inclusion criteria not fulfilled), start of a prohibited concomitant treatment, non-adherence of the subjects to the study schedule (missed or postponed visit), missing data for one or several evaluation criteria, aberrant values, low compliance of the subject to the study product(s) application, premature study end or untraceable subject, or no respect of the constraints envisaged by the protocol. In case of minor protocol deviation, the technician or the investigator repeated the instructions and reminded the subject to follow protocol requirements/study procedures. In case of persistent or major protocol violations, the subject was declared non-compliant and withdrawn from the study because of non-compliance.

The subjects came to the laboratory without having applied any product to the study area since the previous evening. They were informed about the trial objectives, the procedures and the risks of the study, and did sign two copies each of the Consent Form and Information Sheet. The technician verified inclusion and non-inclusion criteria (see Table 2), defined three zones on the forearms according to the randomization list (two treated zones (A and B)) and one non-treated zone (NT), and applied the tested products to the defined zone.

TABLE 2
Inclusion and exclusion criteria.

| Inclusion criteria | Exclusion criteria |
|---|---|
| Sex: female. | Pregnant or nursing woman. |
| Age: between 20 and 45 years, average: 33 years. | Cutaneous pathology, scars, wounds or |
| Phototype: I to IV. | tattoo on the studied zone (forearms). |
| Type: Caucasian. | Use of dermopharmaceutical or cosmetic |
| Subject without hair on the volar aspect of forearms. | product, other than the cleansing product |
| Healthy subject. | on the test zones, the day of the study. |
| Subject having given his/her informed, written consent. | |
| Subject willing to adhere to the protocol and study | |
| procedures. | |

### 3.1.1. Example 1—Anti-Pollution Capacity Study

The protective effect was evaluated by measuring the quantity of particles modelling atmospheric pollution removed from the skin by standardized rinsing. The studied zones were visualized with a video microscope fitted with a mobile, fiber optic ×20 lens, coupled with an image acquisition computer system. The image obtained was recorded on the computer. The surface (in pixels) covered by the particles (black areas) was measured by image analysis using Photoshop Software®. The value obtained on the treated zone was compared to the value obtained on the non-treated zone. The “anti-pollution” power of the tested cosmetic formulations was evaluated by calculating the percentage of skin protection regarding pollutant particles compared to a non-treated zone. It is an in vivo non-invasive and quantitative method.

One single standardized application was carried out by the technician to a skin area of 16 cm², wherein 2 mg/cm² were applied and spread evenly over the skin with a fingerstall.

After 20 minutes waiting, a sample of micronized iron oxide pigment particles (Sunchroma cosmetic pigment, Sun Chemical, USA, d₅₀(vol)=1 μm, simulating fine particles (PM_{2.5})) was applied as pollutant to the defined zones by applying a pressure with a make-up sponge. On the non-treated zone, the skin is moistened with water using a cotton and the pollutant is applied by applying a pressure with a make-up sponge. A picture acquisition of each zone was made using a Hirox® video microscope (t1 before rinsing).

Subsequently, said skin zones were rinsed using a standardized quantity (4 μl/cm²) of water, and wiped with a dry cotton. A further picture acquisition of each zone was made using a Hirox® video microscope (t2 after rinsing).

### Data Analysis

Individual data were presented in raw value tables showing the descriptive statistics: means, medians, minima, maxima, standard errors of the means (SEM) and confidence intervals of 95% (95% CI). Variation tables presented raw variations, percentage variations, descriptive statistics and the results of the statistical analysis (p).

For each subject, the values obtained at t1 (before rinsing) on the treated and the non-treated zones were compared. For each subject, the variations in pixel was calculated according to the following formulas:

$$Δ T = ( {T}_{t 2} - {T}_{t 1} ) \qquad Δ NT = ( {NT}_{t 2} - {NT}_{t 1} ) ,$$

wherein T is the quantity of particles on the zone treated with the tested formulation, NT is quantity of particles on the non-treated zone, t1 is the point in time before rinsing, and t2 is the point in time after rinsing.

TABLE 3
Data analysis.

| | | | | Variations ± | Variations | | Expected |
|---|---|---|---|---|---|---|---|
| | | Parameter | Unit | SEM | in % | p | result |
| Image analysis | Particles | Quantity | pixel | Tₜ₁ − NTₜ₁ | / | yes | no difference |
| with Photoshop ® | adhesion | of particles | | | | | |
| | Protective effect | | pixel | $\frac{t 2 - t 1}{t 1}$ | $\frac{t 2 - t 1}{t 1} \times 100$ | yes | decrease |

In order to determine the protective effect of the tested formulation compared to a non-treated zone, the results are given in percentage of protection (P %). The higher the percentage of protection is, the more efficient the product is. The percentage of protection is calculated according to the following formula:

$$P \% = - [ [ Δ {\%}_{T} = \frac{( {T}_{t 2} - {T}_{t 1} )}{{T}_{t 1}} \times 100 ] - [ Δ {\%}_{N T} = \frac{( N {T}_{t 2} - N {T}_{t 1} )}{N {T}_{t 1}} \times 100 ] ] .$$

TABLE 4
Characterization of protective effect.

| Expected effect | Data | Comparison |
|---|---|---|
| Particle adhesion | t1 | Treated vs. non-treated zone |
| Protective effect | $\frac{t 2 - t 1}{t 1}$ | Treated vs. non-treated zone |

The normality of the differences was determined by the Shapiro-Wilk test (α=0.01). According to the result of the normality test, either the paired student t-test or the Wilcoxon signed rank test was performed. The software used were Excel and SAS 9.4. The analysis conditions are compiled in Table 5 below.

TABLE 5
Analysis conditions for Shapiro-Wilk test.

| Analysis conditions | p-value | H0 | Conclusion |
|---|---|---|---|
| Type I error (α) = 5% in | p ≤ 0.05 | Rejected | Statistically |
| bilateral mode | | | significant difference |
| Null hypothesis (H0) = | p > 0.05 | Not | No statistically |
| no difference between | | rejected | significant difference |
| means or medians | | | |

### Results

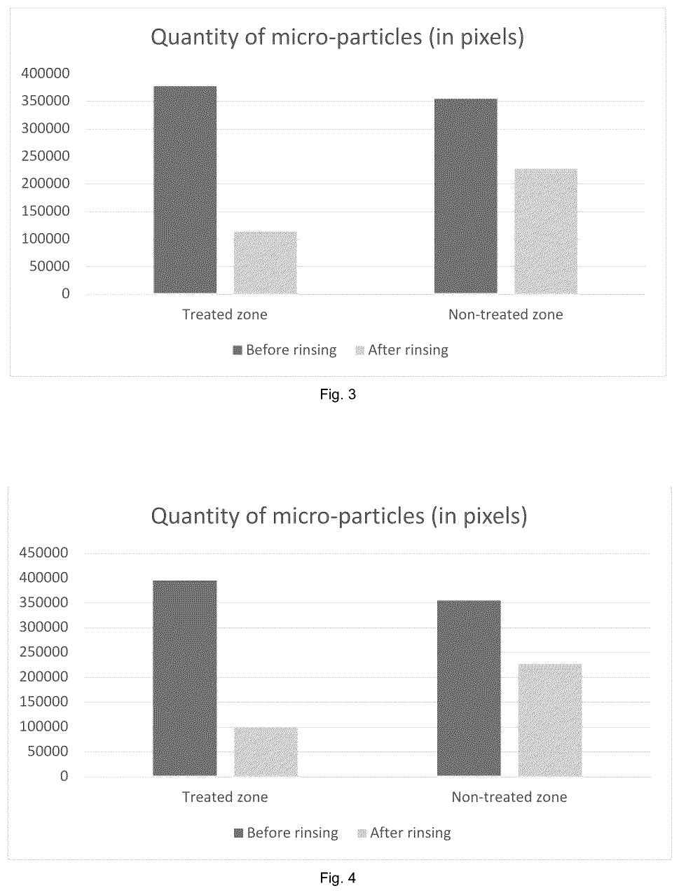

Before cleansing, the formulation 1 showed no difference in terms of adhesion of particles but the formulation 2 held the particles. It means that the formulation 2 was sticky but it still provided a good protective effect. After applying the pollutant, some water was applied on both zones and the difference in quantity of particles removed from the skin between the treated and non-treated zone was measured. In order to determine the protective effect of the product compared to a non-treated zone, the results are given in percentage of protection (P %). The higher the percentage of protection (P %) is, the more efficient the product is. Formulation 1 got a protective effect of 34% and formulation 2 has 39%. The results are compiled in Tables 6, 7 and 8 below as well as in FIGS. 3 and 4.

Both tested cosmetic formulations have a statistically significant protective effect against the particles modelling atmospheric pollution, wherein the protective effect of formulation 2 is better than that of formulation 1.

TABLE 6
Quantity of pollutant particles (in pixels)
deposited to the skin (before rinsing t1).

| | Formulation 1 | Formulation 2 |
|---|---|---|
| | Before rinsing (t1) | Before rinsing (t1) |
| | (mean ± SEM) | (mean ± SEM) |
| Treated zone (A) | 377 000 ± 12 237 | 393 885 ± 13 254 |
| Non-treated zone (NT) | 354 193 ± 14 031 | 354 193 ± 14 031 |
| Δ (A − NT) | 22 808 ± 16 809 | 39 692 ± 17 484 |
| p-value | 0.1899 (student test) | 0.0344 (student test) |

TABLE 7
Variations of the pollutant particles quantity (in pixels).

| | Formulation 1 | | | |
|---|---|---|---|---|
| | | Non-treated | Formulation 2 | Non-treated |
| | Treated zone (A) | zone (NT) | Treated zone (A) | zone (NT) |
| Before rinsing (t1) | 377 000 ± 12 237 | 354 193 ± 14 031 | 393 885 ± 13 254 | 354 193 ± 14 031 |
| (mean ± SEM) | | | | |
| After rinsing (t2) | 113 197 ± 10 069 | 227 376 ± 10 763 | 98 006 ± 6 956 | 227 376 ± 10 763 |
| (mean ± SEM) | | | | |
| ΔT (t2 − t1) | −263 804 ± 13 699 | −126 817 ± 11 639 | −295 880 ± 13 477 | −126 817 ± 11 639 |
| (mean ± SEM) | | | | |
| ΔT % | −70 | −35 | −70 | −35 |
| p-value | <0.0001° (student test) | | <0.0001° (student test) | |
| Percentage of | 34 | | 39 | |
| protection (P %) | | | | |

TABLE 8
Variations of the pollutant particles quantity (in pixels).

| | Formulation 1 | Formulation 2 |
|---|---|---|
| | Treated zone (A) | Treated zone (A) |
| ΔT (mean ± SEM) | −263 804 ± 13 699 | −295 880 ± 13 477 |
| P % | 34 | 39 |

| | |
|---|---|
| P %(formulation 1) − | −5 |
| P %(formulation 2) | |
| p-value | 0.0280 (student test) |

### 3.1.2. Example 2—Cleansing Capacity Study

The cleansing capacity was evaluated by measuring the quantity of particles modelling atmospheric pollution removed from the skin by standardized cleansing. The studied zones were visualized with a video microscope fitted with a mobile, fiber optic ×20 lens, coupled with an image acquisition computer system. The image obtained was recorded on the computer. The surface (in pixels) covered by the particles (black areas) was measured by image analysis using Photoshop Software®. The value obtained on the treated zone was compared to the value obtained on the non-treated zone. The cleansing capacity of tested cosmetic formulations was evaluated by calculating the percentage of cleansing regarding pollutant particles compared to a zone cleansed with water. It is an in vivo non-invasive and quantitative method.

The defined zones were moisturized with a cotton moistened with water and a sample of micronized iron oxide pigment particles (Sunchroma cosmetic pigment, Sun Chemical, USA, d₅₀(vol)=1 μm, simulating fine particles (PM_{2.5})) was applied as pollutant to the defined skin zones by applying a pressure with a make-up sponge. A picture acquisition of each zone was made using a Hirox® video microscope (t1 before rinsing).

Subsequently, one single standardized application was carried out by the technician to the treated skin areas, wherein 4 μl/cm² were applied and massaged into the skin with a fingerstall, and wiped with a dry cotton. On the non-treated zone, 4 μl/cm² water was applied and massaged into the skin with a fingerstall, and wiped with a dry cotton. A further picture acquisition of each zone was made using a Hirox® video microscope (t2 after rinsing).

### Data Analysis

Individual data were presented in raw value tables showing the descriptive statistics: means, medians, minima, maxima, standard errors of the means (SEM) and confidence intervals of 95% (95% CI). Variation tables presented raw variations, percentage variations, descriptive statistics and the results of the statistical analysis (p).

For each subject, the values obtained at t1 (before rinsing) on the treated and the non-treated zones were compared. For each subject, the variations in pixel are calculated according to the following formulas:

$$Δ T = ( {T}_{t 2} - {T}_{t 1} ) \qquad Δ NT = ( {NT}_{t 2} - {NT}_{t 1} ) ,$$

wherein T is the quantity of particles on the zone treated with the studied product, NT is quantity of particles on the non-treated zone, t1 is the point in time before rinsing, and t2 is the point in time after rinsing.

TABLE 9
Data analysis.

| | | | | Variations ± | Variations | | Expected |
|---|---|---|---|---|---|---|---|
| | | Parameter | Unit | SEM | in % | p | result |
| Image analysis | Particles | Quantity | pixel | Tₜ₁ − NTₜ₁ | / | yes | no difference |
| with Photoshop ® | adhesion | of particles | | | | | |
| | Protective effect | | pixel | $\frac{t 2 - t 1}{t 1}$ | $\frac{t 2 - t 1}{t 1} \times 100$ | yes | decrease |

In order to determine the cleansing effect of the formulation compared to a non-treated zone, the results are given in percentage of cleansing (C %). The higher the percentage of cleansing is, the more efficient the formulation is. The percentage of cleansing is calculated according to the following formula:

$$C \% = - [ [ Δ {\%}_{T} = \frac{( {T}_{t 2} - {T}_{t 1} )}{{T}_{t 1}} \times 100 ] - [ Δ {\%}_{N T} = \frac{( N {T}_{t 2} - N {T}_{t 1} )}{N {T}_{t 1}} \times 100 ] ] .$$

TABLE 10
Characterization of protective effect.

| Expected effect | Data | Comparison |
|---|---|---|
| Particle adhesion | t1 | Treated vs. non-treated zone |
| Protective effect | $\frac{t 2 - t 1}{t 1}$ | Treated vs. non-treated zone |

The normality of the differences was determined by the Shapiro-Wilk test (α=0.01). According to the result of the normality test, either the paired student t-test or the Wilcoxon signed rank test was performed. The software used were Excel and SAS 9.4. The analysis conditions are compiled in Table 11 below.

TABLE 11
Analysis conditions for Shapiro-Wilk test.

| Analysis conditions | p-value | H0 | Conclusion |
|---|---|---|---|
| Type I error (α) = 5% in | p ≤ 0.05 | Rejected | Statistically |
| bilateral mode | | | significant difference |
| Null hypothesis (H0) = | p > 0.05 | Not | No statistically |
| no difference between | | rejected | significant difference |
| means or medians | | | |

### Results

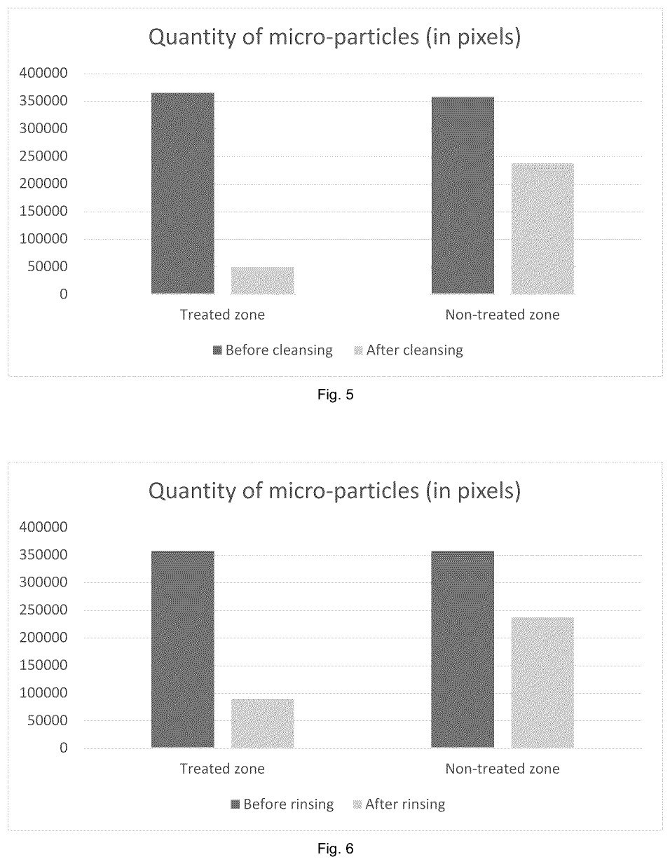

The measurement before cleansing showed that there was no difference in quantity of particles deposed on the skin between the treated and non-treated zone. It means that the quantity of particles deposed was the same on the treated and non-treated zone. After cleaning the quantity of particles removed from the skin was determined between the treated and non-treated zone. The cleansing capacity of the product was determined compared to a zone cleansed with water (non-treated zone), the results are given in percentage of cleansing (C %). The results are compiled in Tables 12, 13 and 14 below as well as in FIGS. 5 and 6.

Both tested cosmetic formulations are evaluated as having a good cleansing effect, wherein the cleansing capacity of formulation 1 is better (C %=53%) than that of formulation 2 (C %=41%).

TABLE 12
Quantity of pollutant particles (in pixels)
deposited to the skin (before rinsing t1).

| | Formulation 1 | Formulation 2 |
|---|---|---|
| | Before rinsing (t1) | Before rinsing (t1) |
| | (mean ± SEM) | (mean ± SEM) |
| Treated zone (A) | 364 519 ± 13 193 | 357 154 ± 11 809 |
| Non-treated zone (NT) | 357 273 ± 13 332 | 357 273 ± 13 332 |
| Δ (A − NT) | 7 247 ± 8 957 | −119 ± 12 607 |
| p-value | 0.4276 (student test) | 0.9926 (student test) |

TABLE 13
Variations of the pollutant particles quantity (in pixels).

| | Formulation 1 | | | |
|---|---|---|---|---|
| | | Non-treated | Formulation 2 | Non-treated |
| | Treated zone (A) | zone (NT) | Treated zone (A) | zone (NT) |
| Before cleansing (t1) | 364 519 ± 13 193 | 357 273 ± 13 332 | 357 154 ± 11 809 | 357 273 ± 13 332 |
| (mean ± SEM) | | | | |
| After cleansing (t2) | 48 154 ± 4 643 | 238 118 ± 15 551 | 89 939 ± 8 498 | 238 118 ± 15 551 |
| (mean ± SEM) | | | | |
| ΔT (t2 − t1) | −316 365 ± 14 214 | −119 154 ± 14 674 | −267 215 ± 16 826 | −119 154 ± 14 674 |
| (mean ± SEM) | | | | |
| ΔT % | −86 | −33 | −74 | −33 |
| p-value | <0.0001° (student test) | | <0.0001° (student test) | |
| Percentage of | 53 | | 41 | |
| cleansing (C %) | | | | |

TABLE 14
Variations of the micro-particles quantity (in pixels).

| | Formulation 1 | Formulation 2 |
|---|---|---|
| | Treated zone (A) | Treated zone (A) |
| ΔT (mean ± SEM) | −316 365 ± 13 699 | −267 215 ± 16 826 |
| P % | 53 | 41 |

| | |
|---|---|
| P %(formulation 1) − | 13 |
| P %(formulation 2) | |
| p-value | 0.0001 (student test) |

### 3.2. Hair Care Compositions

### 3.2.1. Example 3—Sebum Removal Study

### Tested Dry Shampoo Products

DS1: Ground calcium carbonate manufactured from a high purity white marble, d₅₀ (vol)=35 μm, d₉₈ (vol)=150 μm and SSA<1 m²/g, commercially available from Omya International AG.
DS2: SRCC2.
DS3: SRCC1.
DS4: Natural Reisita (rice starch).
DS5: Commercially available dry shampoo (tapioca pure starch (INCI: tapioca starch)).
DS6: Commercially available dry shampoo (Natrasorb HFB starch (INCI: aluminum starch octenylsuccinate (and) acrylates copolymer (and) magnesium carbonate)).

### ATR-FTIR Spectroscopy

The FTIR data were generated with a spotlight system 400 from PerkinElmer with an ATR accessory. The spectra were recorded with the following spectral parameters:
Spectral resolution 4 cm⁻¹
Range 4000-650 cm⁻¹.

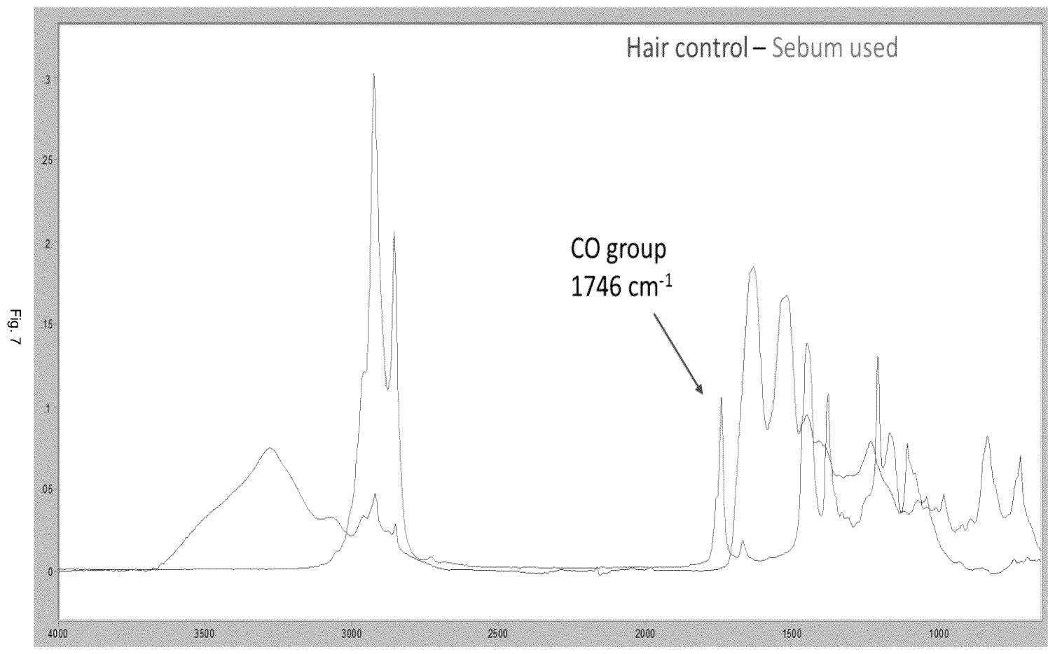

In the FTIR spectra, the position and band intensity give some information about the chemical nature of the material. For example, the contribution of esters always has a carbonyl (C═O) band around 1746 cm⁻¹. This band was used to follow the presence and the reduced content of sebum on the hair tresses after powders application. An FTIR spectra from virgin hair and the artificial sebum solution is shown in FIG. 7.

For each product, the hair samples were tested after sebum application as a positive control and after two powder applications.

To take into consideration the important variation inside the same hair tress, several FTIR spectra were recorded along various parts of the tress (root/middle/tip) for each measurement. The sebum (CO)/protein (Amide I) ratio was defined and calculated to assess the amount of sebum on the hair fibers.

### Experimental Procedure

Caucasian medium brown hair tresses were used as hair samples (supplied by International Hair Importers). Each tress was 8 inches long, 1 inch wide, and weighted approximately 3 g. The tresses were standardized with 0.15 ml of non-conditioning shampoo, massaged, and rinsed under intellifaucet water for 30 seconds each.

0.5 g of an artificial sebum solution (70 wt.-% Olea Europaea (olive) oil, 25 wt.-% squalene, 5 wt.-% wax) was applied on virgin hair tresses to mimic greasy and dirty hair. The tresses were combed with a brush for even distribution of the sebum along the hair fibers (10 brush strokes on front & back of the tress).

125 mg of dry shampoo product was measured out and applied to both sides evenly down the tress, totaling 250 mg of composition per application. After application, the hair tresses were massaged manually for 30 seconds. After 10 minutes the tresses were combed 10 times on each side.

For each product, two total applications were performed on the same hair tresses. For each product, two hair tresses were scanned by ATR-FTIR spectroscopy after sebum application (sebum deposition) and after the second application of powder. For each condition, several spectra were recorded, baseline corrected, and averaged. The carbonyl (C═O) band around 1746 cm-1 was used to follow the removal of sebum on the hair fibers after application of different powders.

### Results

After the application of the sebum onto the hair tresses, a high and uniform sebum deposition was observed on each hair tresses. It was clearly seen, that after two applications of the respective powders, the sebum content on the hair fibers decreased significantly with each powders tested. Hence, all six powders were useful for cleaning dirty hair.

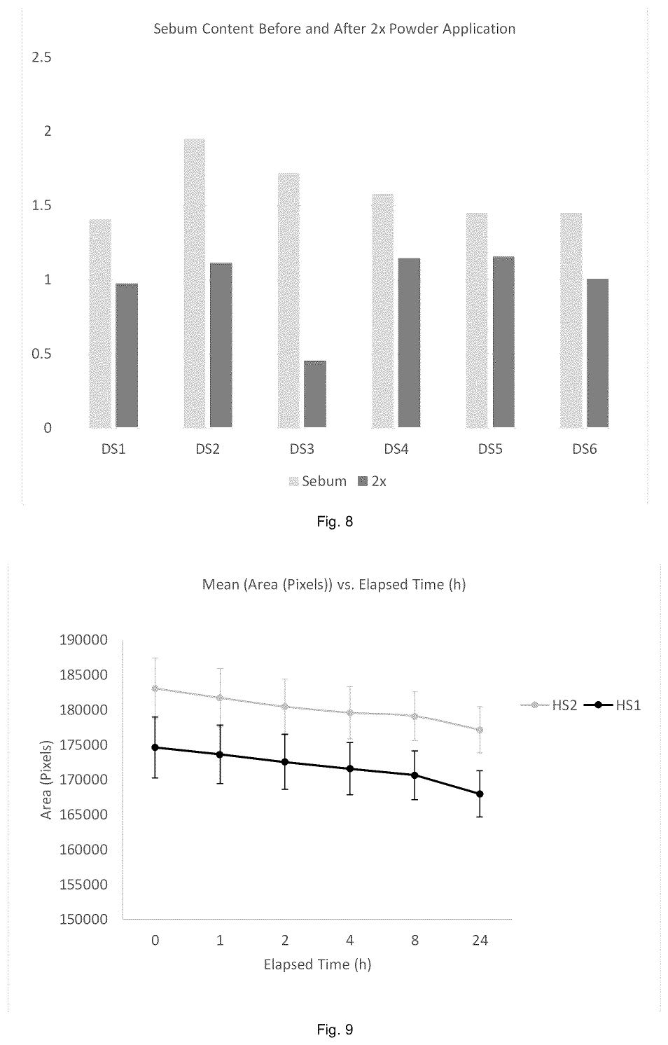

The most efficient powder overall to remove sebum from the hair tresses was the dry shampoo product DS3 consisting of surface reacted calcium carbonate SRCC1. As illustrated in FIG. 8, after the second application the hair tresses treated with product DS3 have a significantly lower amount of sebum compared to the other powders (˜70% of sebum reduction). The dry shampoo product DS1 consisting of GCC and the commercial dry shampoo product DS6 removed comparable amounts of sebum (˜30%) from the hair after two applications. The commercial dry shampoo product DS5 was the least efficient product in terms of sebum removal (only ˜20% of sebum reduction). The results of the ATR-FTIR spectra are compiled in Table 15 below.

TABLE 15
Values related to the ATR-FTIR spectra for
the 1746 to 1645 cm⁻¹ intensity peak ratio.
After sebum application

| Sample | N | Mean | Std Dev | Std Error Mean |
|---|---|---|---|---|
| DS1 | 2 | 1.41 | 0.45 | 0.32 |
| DS2 | 2 | 1.95 | 1.26 | 0.89 |
| DS3 | 2 | 1.72 | 0.79 | 0.56 |
| DS4 | 2 | 1.58 | 0.57 | 0.40 |
| DS5 | 2 | 1.45 | 0.24 | 0.17 |
| DS6 | 2 | 1.45 | 0.24 | 0.17 |

After two product applications

| | | | | | Total percent |
|---|---|---|---|---|---|
| Sample | N | Mean | Std Dev | Std Error Mean | decrease (%) |
| DS1 | 2 | 0.97 | 0.23 | 0.16 | 31.33 |
| DS2 | 2 | 1.11 | 0.42 | 0.30 | 43.12 |
| DS3 | 2 | 0.45 | 0.19 | 0.13 | 73.69 |
| DS4 | 2 | 1.14 | 0.37 | 0.26 | 28.04 |
| DS5 | 2 | 1.15 | 0.39 | 0.28 | 20.27 |
| DS6 | 2 | 1.00 | 0.24 | 0.17 | 30.94 |

### 3.2.2. Example 4—Volume Increase Study

### Tested Hair Styling Products

HS1: SRCC2.

HS2: Ground calcium carbonate manufactured from a high purity white marble, d₅₀ (vol)=35 μm, d₉₈ (vol)=150 μm and SSA<1 m²/g, commercially available from Omya International AG.

### Volume Detection Via Image Analysis

Testing involved the use of an image analysis method to track the changes in tress dimensions and volume before and after treatment with sample products under climate controlled conditions. Prior to all treatments tresses were equilibrated under specified controlled conditions in a climate chamber. High quality photographic images were acquired to characterize the initial state of the tresses for baseline. After treatment with the appropriate product regimes, these hair tresses are again maintained at standard temperature and relative humidity until all samples have been prepared. Tresses were again exposed to standard controlled conditions and additional photographic images were taken at appropriate durations. The volume dimensions were measured from the captured images and determined using custom written TRI software under Lab View™ v2014.

### Experimental Procedure

Eight custom round medium brown hair tresses (6 grams, 8 inches) per treatment group were used as substrates.

For the baseline measurement (sebum treated) 2 g artificial sebum solution per hair tress was applied and worked in with a mascara brush for 20 strokes. All tresses are allowed to dry overnight at 60% relative humidity and ambient temperature before initial images were taken.

250 mg of test product were applied along the hair tresses on each side followed by a 1-minute massage to homogenize the distribution of the powder. After a break of 10 minutes to allow the powder to interact with the sebum, the hair tresses were combed 10 times on each side. All tresses were allowed to equilibrate overnight at 60% relative humidity and ambient temperature. Prior to initial images, tresses were combed to orient for volume testing. Subsequently, the tresses were imaged at 1 hour, 2 hour, 4 hour, 8 hour, and 24 hour time points.

Analysis was completed to compare baseline to initial, 1 hour, 2 hour, 4 hour, 8 hour and 24 hour time points.

### Results

FIG. 9 provides a summary of area (pixels) upon exposure to 60% relative humidity for medium brown hair tresses treated with the test products. Tables 16 and 17 show data collected before and after treatment up to 24 hours of 60% relative humidity exposure.

The medium brown hair tresses treated with test product HS1 compared to sebum treated tresses (baseline) showed a statistically significant increase in volume after each time point. There was no statistically significant difference observed from the initial post-treatment volume of the tresses up to 24 hours post-treatment. Tresses treated with the test product 1 maintained the volume for 24 hours.

The medium brown hair tresses treated with test product HS2 compared to sebum treated tresses (baseline) showed a statistically significant increase in volume after each time point. There was no statistically significant difference observed from the initial post-treatment volume of the tresses up to 24 hours post-treatment. Tresses treated with the test product 2 maintained the volume for 24 hours.

TABLE 16
Area (pixels) of hair before and after
treatment with test product HS1.

| Elapsed | | | | Std Err | | |
|---|---|---|---|---|---|---|
| Time (h) | Number | Mean | Std Dev | Mean | | |
| 0 | 8 | 174575 | 12343 | 4364 | A | |
| 1 | 8 | 173581 | 11819 | 4179 | A | |
| 2 | 8 | 172506 | 11124 | 3933 | A | |
| 4 | 8 | 171542 | 10587 | 3743 | A | |
| 8 | 8 | 170587 | 9876 | 3492 | A | |
| 24 | 8 | 167922 | 9361 | 3310 | A | |
| Baseline | 8 | 26672 | 3420 | 1209 | | B |

TABLE 17
Area (pixels) of hair before and after
treatment with test product HS2.

| Elapsed | | | | Std Err | | |
|---|---|---|---|---|---|---|
| Time (h) | Number | Mean | Std Dev | Mean | | |
| 0 | 8 | 183045 | 13144 | 4647 | A | |
| 1 | 8 | 181695 | 12441 | 4399 | A | |
| 2 | 8 | 180434 | 12303 | 4350 | A | |
| 4 | 8 | 179569 | 12177 | 4305 | A | |
| 8 | 8 | 179061 | 12094 | 4276 | A | |
| 24 | 8 | 177120 | 12459 | 4405 | A | |
| Baseline | 8 | 29180 | 2314 | 818 | | B |

1. A method of using a mineral material as an anti-pollution cosmetic agent comprising the step of adding the mineral material to a cosmetic composition, wherein the mineral material has
a volume median particle size d₅₀(vol) from 0.1 to 90 μm,
a volume top cut particle size d₉₈(vol) of below 250 μm, and
is selected from surface-reacted calcium carbonate, hydromagnesite, or mixtures thereof,
wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

2. The method of claim 1, wherein the mineral material has a volume median particle size d₅₀(vol) from 0.1 to 75 μm and/or a volume top cut particle size d₉₈(vol) from 0.2 to 150 μm.

3. The method of claim 1, wherein the mineral material has an intra-particle intruded specific pore volume in the range from 0.05 to 2.3 cm³/g, calculated from a mercury porosimetry measurement, for a diameter range of 0.004 to 0.8 μm.

4. The method of claim 1, wherein the mineral material has a specific surface area of from 15 m²/g to 200 m²/g, measured using nitrogen and the BET method.

5. The method of claim 1, wherein the natural ground calcium carbonate is selected from the group consisting of marble, chalk, limestone, and mixtures thereof, or the precipitated calcium carbonate is selected from the group consisting of precipitated calcium carbonates having an aragonitic, vateritic or calcitic crystal form, and mixtures thereof.

6. The method of claim 1, wherein the at least one H₃O⁺ ion donor is selected from the group consisting of hydrochloric acid, sulphuric acid, sulphurous acid, phosphoric acid, citric acid, oxalic acid, an acidic salt, acetic acid, formic acid, and mixtures thereof.

7. The method use of claim 1, wherein the mineral material is associated with at least one active agent selected from pharmaceutically active agents, biologically active agents, disinfecting agents, preservatives, flavouring agents, surfactants, oils, fragrances, essential oils, and mixtures thereof.

8. An anti-pollution cosmetic composition comprising a mineral material, wherein the mineral material has
a volume median particle size d50(vol) from 0.1 to 90 μm,
a volume top cut particle size d98(vol) of below 250 μm, and
is selected from surface-reacted calcium carbonate, hydromagnesite, or mixtures thereof,
wherein the surface-reacted calcium carbonate is a reaction product of natural ground calcium carbonate or precipitated calcium carbonate with carbon dioxide and one or more H3O+ ion donors, wherein the carbon dioxide is formed in situ by the H3O+ ion donors treatment and/or is supplied from an external source.

9. The anti-pollution cosmetic composition of claim 8, wherein the mineral material is present in the anti-pollution cosmetic composition in an amount from 0.1 to 50 wt.-%, based on the total weight of the anti-pollution cosmetic composition.

10. The anti-pollution cosmetic composition of claim 8, wherein the anti-pollution cosmetic composition has a pH value of ≤8.5.

11. The anti-pollution cosmetic composition of claim 8, wherein the anti-pollution cosmetic composition further comprises water and/or at least one oil.

12. The anti-pollution cosmetic composition of claim 8, wherein the anti-pollution cosmetic composition further comprises at least one additive selected from the group consisting of bleaching agents, thickeners, stabilizers, chelating agents, preserving agents, wetting agents, emulsifiers, emollients, fragrances, colorants, skin tanning compounds, antioxidants, minerals, pigments, UV-A and/or UV-B filter, and mixtures thereof.

13. The anti-pollution cosmetic composition of claim 8, wherein the anti-pollution cosmetic composition is a sun protection product, an eye make-up product, a facial make-up product, a lip care product, a hair care product, a hair styling product, a hair cleaning product, a nail care product, a hand care product, a hand cleaning product, a skin care product, a skin cleaning product, a scalp care product, a scalp cleaning product, a facial cleaning product, a make-up remover, a facial mist, a cleaning wipe, an exfoliating product, or a combination product thereof.

14. A cosmetic method of protecting a keratin material from pollutants comprising the steps of:
(i) providing an anti-pollution cosmetic composition according to claim 8, and
(ii) applying said anti-pollution cosmetic composition onto the keratin material.

15. The cosmetic method of claim 14, wherein the pollutants are atmospheric pollutants, preferably selected from the group consisting of carbon black, carbon oxides, nitrogen oxides, sulfur oxides, hydrocarbons, organic volatiles, heavy metals, atmospheric particulate matter, fine particulate matter (PM_{2.5}), and mixtures thereof, and/or wherein the keratin material is skin, nails, and/or hair.

16. The method of claim 2, wherein the mineral material has a volume median particle size d₅₀(vol) from 1 to 40 μm, and/or a volume top cut particle size d₉₈(vol) from 2 to 80 μm.

17. The method of claim 16, wherein the mineral material has a volume median particle size d₅₀(vol) from 1.5 to 15 μm, and/or a volume top cut particle size d₉₈(vol) from 3 to 30 μm.

18. The anti-pollution cosmetic composition of claim 9 wherein the mineral material is present in the anti-pollution cosmetic composition in an amount from 3 to 6 wt.-%, based on the total weight of the anti-pollution cosmetic composition.

19. The anti-pollution cosmetic composition of claim 10 wherein the anti-pollution cosmetic composition has a pH value in the range of 4 to 7.

20. The anti-pollution cosmetic composition of claim 11 wherein the at least one oil is selected from the group consisting of vegetable oils and esters thereof, alkanecoconutester, plant extracts, animal fats, siloxanes, silicones, fatty acids and esters thereof, petrolatum, glycerides and pegylated derivatives thereof, and mixtures thereof.
